(19) United States
(12) Patent Application Publication
(10) Pub. No.: US 2024/0093182 A1
(43) Pub. Date: Mar. 21, 2024

(54) EUKARYOTIC DNA REPLICATION ORIGINS, AND VECTOR CONTAINING THE SAME

(71) Applicants: CENTRE NATIONAL DE LA RECHERCHE SCIENTIFIQUE, Paris (FR); UNIVERSITÉ DE MONTPELLIER, Montpellier (FR)

(72) Inventors: Marcel MECHALI, Montferrier-sur-Lez (FR); Ildem AKERMAN, Birmingham (GB); Nadège GABORIT, Valflaunes (FR)

(73) Assignees: CENTRE NATIONAL DE LA RECHERCHE SCIENTIFIQUE, Paris (FR); UNIVERSITÉ DE MONTPELLIER, Montpellier (FR)

(21) Appl. No.: 18/041,902

(22) PCT Filed: Sep. 6, 2021
(86) PCT No.: PCT/EP2021/074523

§ 371 Date: Feb. 16, 2023

(30) Foreign Application Priority Data
Sep. 7, 2020 (EP) 20305987.8

(51) Int. Cl. C12N 15/10; C12N 15/85

(52) U.S. Cl. CPC C12N 15/1093; C12N 15/85; C12N 2820/85

(57) ABSTRACT

A method for isolating a mammalian genomic DNA replication origin, the method including: isolating the genomic DNA molecules; identifying 500 bp windows within the DNA molecules; isolating from the genomic DNA molecules the fragments that have a size from 500 pb up 6000 pb; selecting a DNA replication origin that is able, when contained in the DNA of an Eukaryotic cell, to produce nascent DNA, and to initiate DNA replication; and isolating the origin.

The invention relates to eukaryotic DNA replication origins and vector containing the same.

During each cell division, a human cell will replicate approximately two meters of DNA within the S-phase time constraints. To achieve this, DNA replication initiates from thousands of regions that are called DNA replication origins and are spread across the genome. The positioning of DNA replication initiation sites (IS) in the genome (origin specification) is poorly understood in metazoans. In prokaryotes and viruses, usually a single, sequence-specific origin exists, while in the eukaryote Saccharomyces cerevisiae, DNA replication initiates from AT-rich consensus sequences that are bound by the yeast origin recognition complex (ORC). By contrast, in fruit fly and mouse cells, the presence of a G-rich DNA sequence element, (Origin G-rich Repeated Element, OGRE), around 300 bp upstream of the IS has been reported in more than 60% of origins. CA/GT-rich motifs and poly-A/T tracks have also been detected at IS in mouse cells. OGRE elements may contain CpG islands (CpGi) and potential G-quadruplex (G4) elements, in a nucleosome-free region. However, only a fraction of all putative G4 elements in the genome host a nearby origin, and CpGi are present only in a fraction of origins. This indicates that other features contribute to replication origin selection or activation.

So there is a need to better understand how a replication origin works, and how to identify them.

Some information is known in the mouse regarding the mammalian replication origins.

For instance, international application WO2011023827 discloses sequence of replication origin core, and in particular the OGRE sequences. But this document fails to disclose the sequence of fully functional replication origins or origins in the human genome.

So one aim of the invention is obviate this drawback.

Another aim of the invention is to provide a method for identifying and isolating the functional DNA sequences that can self-replicate, in an appropriated context.

A further aim of the invention is to provide a DNA vector that can replicate in a host mammalian cell as the chromosome does, since these vectors contain a functional mammalian replication origin.

Thus, the invention relates to a method for isolating a mammalian genomic DNA replication origin, the method comprising:
a—isolating the genomic DNA molecules from a somatic cell of a mammal;
b—dividing the genomic DNA molecules into 500 bp windows every 100 pb along said genomic DNA molecules,
c—identifying a first 500 bp windows such that:
   the first 500 bp window has at least 172 G nucleotides,
   the first 500 bp window has no more than 105 A or T nucleotides,
   a second 500 bp window immediately adjacent to the first 500 bp window at the 3′-end of the window has a G content lower than the 172 and higher than 125;
   wherein the variation of the G content between the first and the second 500 bp window is ranging from 8% to 40%;
   the G content in a large window consisting of 8 consecutive 500 bp-windows constituted by a third 500 bp windows adjacent to a fourth 500 bp windows, itself adjacent to a fifth 500 bp windows, itself adjacent to the first 500 bp windows, itself adjacent to the second 500 bp windows, itself adjacent to a sixth 500 bp windows, itself adjacent to a seventh 500 bp windows, itself adjacent to a eighth 500 bp windows, is higher than 960;
d—isolating from the genomic DNA molecules the fragments that have a size from 500 bp up 6000 bp corresponding to putative mammalian genomic DNA replication origin, wherein the putative mammalian genomic DNA replication origin consists at its 5′end of the first 500 bp window,
e—selecting from said putative mammalian genomic DNA replication origin a fragment that is able, when contained in the DNA of a Eukaryotic cell, to produce nascent DNA, and to initiate DNA replication; and
f—Isolating said fragment, wherein said fragment is a mammalian genomic DNA replication origin.

The invention is based on the observation made by the inventors that the core DNA replication origins can be identified and isolated by implementing the above-mentioned described method.

This method allows to identify the mammalian replication origins that are fully active and present in all the mammal genomes.

The method according to the invention is carried out in two steps: a step of identifying the core origin sequence, and a step selecting the sequence that match with experimental data.

Step a).

In step A, the genomic DNA of a mammalian cell is extracted according to one method well known in art, such as phenol/chloroform method, sequenced and bioinformatically assembled.

Otherwise, the sequence of the genome as published in database can be used in order to carry out step a. For instance, for mouse and human genomes and others the complete sequence of the genome is available on University of California, Santa Cruz (UCSC) genome browser (available at https://genome.ucsc.edu):

The skilled person could adapt the extraction of DNA for that purpose.

Step b) and c)

These two steps correspond to the identification step.

Step b) is carried out after having obtained the sequence of the DNA molecules contained in the mammal cells. For that purpose, any sequencing technique can be used in order to obtain the complete sequence of the DNA molecules, i.e. the complete sequences of the DNA of each chromosome contained in a mammal cell. This will be followed by assembly of the DNA sequences to obtain the full sequence of a genome.

After having obtained the sequence, the sequences are divided into 500 bp windows every 100 bp along the molecules (also known as the sliding windows method). This is done both for the Watson and the Crick strand.

For instance, in a 1000 bp molecule, six 500 pb windows can be obtained: from position 1 to position 500, from position 100 to position 600, from position 200 to position 700, from position 300 to position 800, from position 400 to position 900 and from position 500 to position 1000. In the full human genome, many 500 bp can be therefore generated.

This step can be easily carried out by a computer program, for instance bedtools suite.

Step c is formally the step of selection of the sequences of interest. The inventors identify that the replication origins in mammal contain a 500 bp region that meet the following criteria:
a 500 bp window of interest has at least 172 G nucleotides, and no more than 105 A or T nucleotides,
when considering a determined 500 bp window, the immediately adjacent 500 bp window that starts at the 3′-end of the 500 pb the determined window has a G content lower than the 172 and higher than 125; wherein the variation of the G content between a determined 500 bp window and its adjacent window is ranging from 8% to 40%. Here this means that if the 500 bp window contains 172 bp, then the G content of the adjacent region varies from 125 to 158 (in fact from 105 to 158, but since the G content shall be higher than 125, the range is 125 to 158); and
in a large window consisting of 8 consecutive 500 bp-windows constituted by a third 500 bp windows adjacent to a fourth 500 bp windows, itself adjacent to a fifth 500 bp windows, itself adjacent to the first 500 bp windows, itself adjacent to the second 500 bp windows, itself adjacent to a sixth 500 bp windows, itself adjacent to a seventh 500 bp windows, itself adjacent to a eighth 500 bp windows, the average G content along the 8 consecutive windows is higher than 960.

As mentioned in the example, the inventors identified that the replication origins in mammals, despite they do not share a stricto sensu consensus sequence, are characterized in that in 5′ of the initiation site of the transcription a 500 pb G-rich region is present, and in 3′ of the initiation site, the region is not a G-rich region. This is clearly illustrated in FIG. 72, left panel.

Here again, this step can be carried out by a computer program.

After having identified, along the genome of a mammal cell, all the 500 bp windows that meet the above criteria, step d) is carried out.

Step d)

In step d), when the 500 bp windows of interest have been identified, fragments of the genome that have a size from 500 pb to 6000 bp are selected. These fragments correspond to the molecules of DNA that may contain a replication origin. They are called “putative replication origins”.

By “from 500 pb to 6000 bp”, it is meant in the invention molecules having a size of 500 bp, 510 bp, 520 bp, 530 bp, 540 bp, 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp, 930 bp, 940 bp, 950 bp, 960 bp, 970 bp, 980 bp, 990 bp, 1000 bp, 1010 bp, 1020 bp, 1030 bp, 1040 bp, 1050 bp, 1060 bp, 1070 bp, 1080 bp, 1090 bp, 1100 bp, 1110 bp, 1120 bp, 1130 bp, 1140 bp, 1150 bp, 1160 bp, 1170 bp, 1180 bp, 1190 bp, 1200 bp, 1210 bp, 1220 bp, 1230 bp, 1240 bp, 1250 bp, 1260 bp, 1270 bp, 1280 bp, 1290 bp, 1300 bp, 1310 bp, 1320 bp, 1330 bp, 1340 bp, 1350 bp, 1360 bp, 1370 bp, 1380 bp, 1390 bp, 1400 bp, 1410 bp, 1420 bp, 1430 bp, 1440 bp, 1450 bp, 1460 bp, 1470 bp, 1480 bp, 1490 bp, 1500 bp, 1510 bp, 1520 bp, 1530 bp, 1540 bp, 1550 bp, 1560 bp, 1570 bp, 1580 bp, 1590 bp, 1600 bp, 1610 bp, 1620 bp, 1630 bp, 1640 bp, 1650 bp, 1660 bp, 1670 bp, 1680 bp, 1690 bp, 1700 bp, 1710 bp, 1720 bp, 1730 bp, 1740 bp, 1750 bp, 1760 bp, 1770 bp, 1780 bp, 1790 bp, 1800 bp, 1810 bp, 1820 bp, 1830 bp, 1840 bp, 1850 bp, 1860 bp, 1870 bp, 1880 bp, 1890 bp, 1900 bp, 1910 bp, 1920 bp, 1930 bp, 1940 bp, 1950 bp, 1960 bp, 1970 bp, 1980 bp, 1990 bp, 2000 bp, 2010 bp, 2020 bp, 2030 bp, 2040 bp, 2050 bp, 2060 bp, 2070 bp, 2080 bp, 2090 bp, 2100 bp, 2110 bp, 2120 bp, 2130 bp, 2140 bp, 2150 bp, 2160 bp, 2170 bp, 2180 bp, 2190 bp, 2200 bp, 2210 bp, 2220 bp, 2230 bp, 2240 bp, 2250 bp, 2260 bp, 2270 bp, 2280 bp, 2290 bp, 2300 bp, 2310 bp, 2320 bp, 2330 bp, 2340 bp, 2350 bp, 2360 bp, 2370 bp, 2380 bp, 2390 bp, 2400 bp, 2410 bp, 2420 bp, 2430 bp, 2440 bp, 2450 bp, 2460 bp, 2470 bp, 2480 bp, 2490 bp, 2500 bp, 2510 bp, 2520 bp, 2530 bp, 2540 bp, 2550 bp, 2560 bp, 2570 bp, 2580 bp, 2590 bp, 2600 bp, 2610 bp, 2620 bp, 2630 bp, 2640 bp, 2650 bp, 2660 bp, 2670 bp, 2680 bp, 2690 bp, 2700 bp, 2710 bp, 2720 bp, 2730 bp, 2740 bp, 2750 bp, 2760 bp, 2770 bp, 2780 bp, 2790 bp, 2800 bp, 2810 bp, 2820 bp, 2830 bp, 2840 bp, 2850 bp, 2860 bp, 2870 bp, 2880 bp, 2890 bp, 2900 bp, 2910 bp, 2920 bp, 2930 bp, 2940 bp, 2950 bp, 2960 bp, 2970 bp, 2980 bp, 2990 bp, 3000 bp, 3010 bp, 3020 bp, 3030 bp, 3040 bp, 3050 bp, 3060 bp, 3070 bp, 3080 bp, 3090 bp, 3100 bp, 3110 bp, 3120 bp, 3130 bp, 3140 bp, 3150 bp, 3160 bp, 3170 bp, 3180 bp, 3190 bp, 3200 bp, 3210 bp, 3220 bp, 3230 bp, 3240 bp, 3250 bp, 3260 bp, 3270 bp, 3280 bp, 3290 bp, 3300 bp, 3310 bp, 3320 bp, 3330 bp, 3340 bp, 3350 bp, 3360 bp, 3370 bp, 3380 bp, 3390 bp, 3400 bp, 3410 bp, 3420 bp, 3430 bp, 3440 bp, 3450 bp, 3460 bp, 3470 bp, 3480 bp, 3490 bp, 3500 bp, 3510 bp, 3520 bp, 3530 bp, 3540 bp, 3550 bp, 3560 bp, 3570 bp, 3580 bp, 3590 bp, 3600 bp, 3610 bp, 3620 bp, 3630 bp, 3640 bp, 3650 bp, 3660 bp, 3670 bp, 3680 bp, 3690 bp, 3700 bp, 3710 bp, 3720 bp, 3730 bp, 3740 bp, 3750 bp, 3760 bp, 3770 bp, 3780 bp, 3790 bp, 3800 bp, 3810 bp, 3820 bp, 3830 bp, 3840 bp, 3850 bp, 3860 bp, 3870 bp, 3880 bp, 3890 bp, 3900 bp, 3910 bp, 3920 bp, 3930 bp, 3940 bp, 3950 bp, 3960 bp, 3970 bp, 3980 bp, 3990 bp, 4000 bp, 4010 bp, 4020 bp, 4030 bp, 4040 bp, 4050 bp, 4060 bp, 4070 bp, 4080 bp, 4090 bp, 4100 bp, 4110 bp, 4120 bp, 4130 bp, 4140 bp, 4150 bp, 4160 bp, 4170 bp, 4180 bp, 4190 bp, 4200 bp, 4210 bp, 4220 bp, 4230 bp, 4240 bp, 4250 bp, 4260 bp, 4270 bp, 4280 bp, 4290 bp, 4300 bp, 4310 bp, 4320 bp, 4330 bp, 4340 bp, 4350 bp, 4360 bp, 4370 bp, 4380 bp, 4390 bp, 4400 bp, 4410 bp, 4420 bp, 4430 bp, 4440 bp, 4450 bp, 4460 bp, 4470 bp, 4480 bp, 4490 bp, 4500 bp, 4510 bp, 4520 bp, 4530 bp, 4540 bp, 4550 bp, 4560 bp, 4570 bp, 4580 bp, 4590 bp, 4600 bp, 4610 bp, 4620 bp, 4630 bp, 4640 bp, 4650 bp, 4660 bp, 4670 bp, 4680 bp, 4690 bp, 4700 bp, 4710 bp, 4720 bp, 4730 bp, 4740 bp, 4750 bp, 4760 bp, 4770 bp, 4780 bp, 4790 bp, 4800 bp, 4810 bp, 4820 bp, 4830 bp, 4840 bp, 4850 bp, 4860 bp, 4870 bp, 4880 bp, 4890 bp, 4900 bp, 4910 bp, 4920 bp, 4930 bp, 4940 bp, 4950 bp, 4960 bp, 4970 bp, 4980 bp, 4990 bp, 5000 bp, 5010 bp, 5020 bp, 5030 bp, 5040 bp, 5050 bp, 5060 bp, 5070 bp, 5080 bp, 5090 bp, 5100 bp, 5110 bp, 5120 bp, 5130 bp, 5140 bp, 5150 bp, 5160 bp, 5170 bp, 5180 bp, 5190 bp, 5200 bp, 5210 bp, 5220 bp, 5230 bp, 5240 bp, 5250 bp, 5260 bp, 5270 bp, 5280 bp, 5290 bp, 5300 bp, 5310 bp, 5320 bp, 5330 bp, 5340 bp, 5350 bp, 5360 bp, 5370 bp, 5380 bp, 5390 bp, 5400 bp, 5410 bp, 5420 bp, 5430 bp, 5440 bp, 5450 bp, 5460 bp, 5470 bp, 5480 bp, 5490 bp, 5500 bp, 5510 bp, 5520 bp, 5530 bp, 5540 bp, 5550 bp, 5560 bp, 5570 bp, 5580 bp, 5590 bp, 5600 bp, 5610 bp, 5620 bp, 5630 bp, 5640 bp, 5650 bp, 5660 bp, 5670 bp, 5680 bp, 5690 bp, 5700 bp, 5710 bp, 5720 bp, 5730 bp, 5740 bp, 5750 bp, 5760 bp, 5770 bp, 5780 bp, 5790 bp, 5800 bp, 5810 bp, 5820 bp, 5830 bp, 5840 bp, 5850 bp, 5860 bp, 5870 bp, 5880 bp, 5890 bp, 5900 bp, 5910 bp, 5920 bp, 5930 bp, 5940 bp, 5950 bp, 5960 bp, 5970 bp, 5980 bp, 5990 bp or 6000 bp.

Step e)

From the molecules selected in step d), only are retained the molecules that produce nascent DNA, and initiate DNA replication. For this purpose, the regions of the genome that produce nascent DNA (i.e. the small molecules that are synthesized when the origin loop is opened) is identified through experimental procedures detailed below:

Identification of Nascent DNA is well known in the art, and it can be carried out by using the SNS-seq protocol as described in the example below (see Nascent strand isolation (SNS-seq)).

If a fragment isolated at step d is overlapping (at least 1 bp) with the nascent DNA that is experimentally identified, then the fragment contains, or corresponds to, a replication origin according to the invention.

Therefore, fragments that share all the above-mentioned criteria are true and accurate replication origin of mammal cells, and if these fragments are inserted in the genome of a mammal cell, or if they are placed in presence of all the proteins necessary for initiating DNA replication, then a replication will occur from these fragments.

Step f)

This step is a step of isolating the fragment of interest, for instance for cloning purpose or for further studies.

in the invention, mammals refer in particular to rodent and human, more preferably mice and humans.

According to the invention, step d) and step e) can be inverted. Therefore the method comprises the steps of:
a—isolating the genomic DNA molecules from a somatic cell of a mammal;
b—dividing the genomic DNA molecules into 500 bp windows every 100 pb along said genomic DNA molecules,
c—identifying a first 500 bp windows such that:
   the first 500 bp window has at least 172 G nucleotides,
   the first 500 bp window has no more than 105 A or T nucleotides,
   a second 500 bp window immediately adjacent to the first 500 bp window at the the 3′-end of the window has a G content lower than the 172 and higher than 125;
   wherein the variation of the G content between the first and the second 500 bp window is ranging from 8% to 40%;
   the G content in a large window consisting of 8 consecutive 500 bp-windows constituted by a third 500 bp windows adjacent to a fourth 500 bp windows, itself adjacent to a fifth 500 bp windows, itself adjacent to the first 500 bp windows, itself adjacent to the second 500 bp windows, itself adjacent to a sixth 500 bp windows, itself adjacent to a seventh 500 bp windows, itself adjacent to a eighth 500 bp windows, is higher than 960;
d—identifying in the whole genome of the somatic cell of a mammal the DNA molecules that are able to produce nascent DNAs, and to initiate DNA replication, said molecules having a size ranging from 500 bp up to 6000 bp and being putative mammalian genomic DNA replication origin;
e—selecting from said putative mammalian genomic DNA replication origins the DNA molecules that consist at their 5′end of the first 500 bp window and which are mammalian genomic DNA replication origin; and
f—Isolating mammalian genomic DNA replication origins.

Advantageously, the invention relates to the method mentioned above, wherein said putative mammalian genomic DNA replication origin have size varying from 500 bp to 4000 bp.

By “from 500 pb to 4000 bp”, it is meant in the invention molecules having a size of 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp, 930 bp, 940 bp, 950 bp, 960 bp, 970 bp, 980 bp, 990 bp, 1000 bp, 1010 bp, 1020 bp, 1030 bp, 1040 bp, 1050 bp, 1060 bp, 1070 bp, 1080 bp, 1090 bp, 1100 bp, 1110 bp, 1120 bp, 1130 bp, 1140 bp, 1150 bp, 1160 bp, 1170 bp, 1180 bp, 1190 bp, 1200 bp, 1210 bp, 1220 bp, 1230 bp, 1240 bp, 1250 bp, 1260 bp, 1270 bp, 1280 bp, 1290 bp, 1300 bp, 1310 bp, 1320 bp, 1330 bp, 1340 bp, 1350 bp, 1360 bp, 1370 bp, 1380 bp, 1390 bp, 1400 bp, 1410 bp, 1420 bp, 1430 bp, 1440 bp, 1450 bp, 1460 bp, 1470 bp, 1480 bp, 1490 bp, 1500 bp, 1510 bp, 1520 bp, 1530 bp, 1540 bp, 1550 bp, 1560 bp, 1570 bp, 1580 bp, 1590 bp, 1600 bp, 1610 bp, 1620 bp, 1630 bp, 1640 bp, 1650 bp, 1660 bp, 1670 bp, 1680 bp, 1690 bp, 1700 bp, 1710 bp, 1720 bp, 1730 bp, 1740 bp, 1750 bp, 1760 bp, 1770 bp, 1780 bp, 1790 bp, 1800 bp, 1810 bp, 1820 bp, 1830 bp, 1840 bp, 1850 bp, 1860 bp, 1870 bp, 1880 bp, 1890 bp, 1900 bp, 1910 bp, 1920 bp, 1930 bp, 1940 bp, 1950 bp, 1960 bp, 1970 bp, 1980 bp, 1990 bp, 2000 bp, 2010 bp, 2020 bp, 2030 bp, 2040 bp, 2050 bp, 2060 bp, 2070 bp, 2080 bp, 2090 bp, 2100 bp, 2110 bp, 2120 bp, 2130 bp, 2140 bp, 2150 bp, 2160 bp, 2170 bp, 2180 bp, 2190 bp, 2200 bp, 2210 bp, 2220 bp, 2230 bp, 2240 bp, 2250 bp, 2260 bp, 2270 bp, 2280 bp, 2290 bp, 2300 bp, 2310 bp, 2320 bp, 2330 bp, 2340 bp, 2350 bp, 2360 bp, 2370 bp, 2380 bp, 2390 bp, 2400 bp, 2410 bp, 2420 bp, 2430 bp, 2440 bp, 2450 bp, 2460 bp, 2470 bp, 2480 bp, 2490 bp, 2500 bp, 2510 bp, 2520 bp, 2530 bp, 2540 bp, 2550 bp, 2560 bp, 2570 bp, 2580 bp, 2590 bp, 2600 bp, 2610 bp, 2620 bp, 2630 bp, 2640 bp, 2650 bp, 2660 bp, 2670 bp, 2680 bp, 2690 bp, 2700 bp, 2710 bp, 2720 bp, 2730 bp, 2740 bp, 2750 bp, 2760 bp, 2770 bp, 2780 bp, 2790 bp, 2800 bp, 2810 bp, 2820 bp, 2830 bp, 2840 bp, 2850 bp, 2860 bp, 2870 bp, 2880 bp, 2890 bp, 2900 bp, 2910 bp, 2920 bp, 2930 bp, 2940 bp, 2950 bp, 2960 bp, 2970 bp, 2980 bp, 2990 bp, 3000 bp, 3010 bp, 3020 bp, 3030 bp, 3040 bp, 3050 bp, 3060 bp, 3070 bp, 3080 bp, 3090 bp, 3100 bp, 3110 bp, 3120 bp, 3130 bp, 3140 bp, 3150 bp, 3160 bp, 3170 bp, 3180 bp, 3190 bp, 3200 bp, 3210 bp, 3220 bp, 3230 bp, 3240 bp, 3250 bp, 3260 bp, 3270 bp, 3280 bp, 3290 bp, 3300 bp, 3310 bp, 3320 bp, 3330 bp, 3340 bp, 3350 bp, 3360 bp, 3370 bp, 3380 bp, 3390 bp, 3400 bp, 3410 bp, 3420 bp, 3430 bp, 3440 bp, 3450 bp, 3460 bp, 3470 bp, 3480 bp, 3490 bp, 3500 bp, 3510 bp, 3520 bp, 3530 bp, 3540 bp, 3550 bp, 3560 bp, 3570 bp, 3580 bp, 3590 bp, 3600 bp, 3610 bp, 3620 bp, 3630 bp, 3640 bp, 3650 bp, 3660 bp, 3670 bp, 3680 bp, 3690 bp, 3700 bp, 3710 bp, 3720 bp, 3730 bp, 3740 bp, 3750 bp, 3760 bp, 3770 bp, 3780 bp, 3790 bp, 3800 bp, 3810 bp, 3820 bp, 3830 bp, 3840 bp, 3850 bp, 3860 bp, 3870 bp, 3880 bp, 3890 bp, 3900 bp, 3910 bp, 3920 bp, 3930 bp, 3940 bp, 3950 bp, 3960 bp, 3970 bp, 3980 bp, 3990 bp, 4000 bp.

Advantageously, the invention relates to the method mentioned above, wherein the 500 bp window of a fragment interacts with ORC1 or ORC2 replication initiation factors.

The first step in the initiation of eukaryotic DNA replication is the assembly of a six-subunit origin recognition complex (ORC) at specific sites distributed throughout the genome at the replication origin.

Whereas the DNA sequence that specifically interact with ORC proteins is not known, it is possible to determine if a DNA molecule interact with ORC proteins, in particular ORC1 or ORC2, or both, by many techniques well known in the art, such as Chromatin IP (ChIP experiments or ChIP-seq) or DNA footprinting, Electrophoretic Mobility Shift Assay . . . .

More advantageously, the invention relates to the method mentioned above, wherein sequence immediately adjacent to the 500 pb window contains:
either multiple tandemly G4 structures, wherein said tandemly G4 structures are present up to 12 times, or
G-rich Repeated Element, or OGRE, or
both.

Advantageously, the replication origins according to the invention may contain G4 structures that are tandemly repeated up to 12 times.

G-quadruplex secondary structures (G4) are formed in nucleic acids by sequences that are rich in guanine. These structures are helical in shape and contain guanine tetrads that can form from one, two or four strands. The unimolecular forms often occur naturally near the ends of the chromosomes, better known as the telomeric regions, and in transcriptional regulatory regions of multiple genes. Four guanine bases can associate through Hoogsteen hydrogen bonding to form a square planar structure called a guanine tetrad (G-tetrad or G-quartet), and two or more guanine tetrads (from G-tracts, continuous runs of guanine) can stack on top of each other to form a G-quadruplex.

The position and bonding to form G-quadruplexes is not random and serve very unusual functional purposes and are located closed to replication origins.
the replication origins according to the invention may alternatively, or additionally contain G-rich Repeated Element, or OGRE, as defined in the international application WO2011023827.

More advantageously, the invention relates to the method mentioned above, wherein the fragment contains a 716 pb (average size) core initiation origin sequence, the core initiation origin sequence being complementary to nascent DNA fragments sequence.

This sequence of about 716 pb (which corresponds to an average size) core initiation origin sequence is the region where the DNA polymerase synthesizes the first RNA-primed nascent strands after the opening of the double strand helix.

More advantageously, the invention relates to the method mentioned above, wherein the fragment also contains binding sites for polycomb proteins or open chromatin such as driven by histone acetylation marks, or both.

DNA methylation, histone modifications, and chromatin configuration are crucially important in the regulation of gene expression. Histone acetylation marks may include H3 and H4 acetylation. Among these epigenetic mechanisms, Polycomb (Pc) proteins play roles in gene silencing through different mechanisms. These proteins act in complexes and govern the histone methylation profiles of a large number of genes that regulate various cellular pathways. They are also associated with replication origin sites.

For instance, histone 3 K27 acetylation is a histone mark commonly associated with enhancer function and to mark active enhancers.

The invention also relates to a mammalian genomic DNA replication origin liable to be obtained, or directly obtained by the method as defined above.

Advantageously, the invention relates to the mammalian genomic DNA replication origin as defined above, the mammalian genomic DNA replication origin comprising one of the sequences as set forth in SEQ ID NO: 1 and SEQ ID NO: 3 to SEQ ID NO: 43,177 and in SEQ ID NO: 43,220 to 43,288.

All these sequences correspond to DNA core origins of mammals. These sequences are novel. The DNA molecule as set forth in the above-mentioned sequences are isolated from their natural context and purified.

It is obviously understood in the invention that “SEQ ID NO: 1 to SEQ ID NO: 43,177 and in SEQ ID NO: 43,220 to 43,288” means that all the 43246 sequences are disclosed, in particular in the attached sequence listing.

Advantageously, the invention relates to the mammalian genomic DNA replication origin as defined above, the mammalian genomic DNA replication origin consisting of one of the sequences as set forth in SEQ ID NO: 1 to SEQ ID NO: 43,177 and in SEQ ID NO: 43,220 to 43,288.

By “SEQ ID NO: 1 to SEQ ID NO: 43177 and in SEQ ID NO: 43,220 to 43,288.” it is meant in the invention all the sequences from SEQ ID NO:1 to SEQ ID NO:43177 and in SEQ ID NO: 43,220 to 43,288 as disclosed in the sequence listing annexed to this description.

These sequences correspond to core origins of mammal DNA molecules, i.e. sequences from which initiation of DNA replication is possible. When inserted in the genome of a [hypothetical] mammalian cell devoid of replication origin, these sequences can promote a new genomic replication origin, i.e. opening of the double strand, neosynthesis of complementary DNA . . . . They can also promote autonomous DNA replication when inserted in a plasmid.

The invention also relates to a vector comprising:
a mammalian genomic DNA replication origin as defined above,
at least a sequence coding for a protein allowing the resistance or sensitivity to a compound specific to eukaryotic cells, and
a region independent to the mammalian genomic DNA replication origin allowing to insert a gene of interest and its expression.

The vector according to the invention contains at least a mammalian replication origin capable of replication in a variety of host mammal cells. This replication is due to the presence of the core origin as defined above.

This vector contains also a region independent to the replication origin were a gene can be inserted, in particular a gene of interest for instance for therapeutic purpose. The region independent to the mammalian genomic DNA replication origin is in particular a cloning site that allows insertion of a nucleic acid sequence of interest, such as a gene of interest or a sequence allowing an epigenetic modification. Advantageously, the cloning site(s) comprise at least one restriction site, i.e., a site where the vector may be selectively cleaved by a particular enzyme. Such sites are known to those skilled in the art. The restriction site may be a unique restriction site, i.e., a restriction site not found elsewhere in the vector or nucleic acid sequence of interest. The cloning site of the vector may comprise a plurality of unique restriction sites to permit insertion of a wide variety of nucleic acid sequences. Illustrative examples of restriction sites include, but are not limited to, the following: HindIII site, BamHI site, Asp718I site, Kpn I site, Bst I site, EcoRI site, EcoRV site, PstI site, Eco32I site, XhoI site, Sfr274I site, XbaI site, FauNDI site, NdeI site, and PmeI site.

In other words, the invention does not encompass vectors were a genomic DNA fragment containing a mammalian replication origin has been cloned into the vector in the cloning site.

The vector also contains a gene, placed under the control of the appropriated means allowing its transcription and the expression of the corresponding protein, the gene coding for a protein that confers either resistance or sensibility to a drug that specifically target eukaryotic cells. This corresponds to a marker gene.

The vector may also possibly contain an inducible transcription promoter able to promote transcription close or through the replication origin.

Marker genes conferring resistance to a drug are well known in the and can be for instance: Zeomycin resistance gene, Neomycin resistance gene, Bleomycin resistance gene, Puromycin resistance gene . . . . Genes conferring sensibility are traditionally those encoding enzymes lacking in the recipient cell, such as HPRT, thymidine kinase, dihydrofolate reductase and APRT. More recently, other genes, such as XGPT, metallothioneine and methotrexate-resistant DHFR, have been employed, as they confer new characteristics on the recipient. This list is not limitative, and the skilled person would easily use the appropriated selection marker gene according to the experiments he would carry out (resistance gene for isolating specific clone, sensitivity gene for killing transfected/transformed cells).

Advantageously, the above mentioned vector is the vector as set forth in SEQ ID NO: 43,389, in which is inserted one of the sequences as set forth in SEQ ID NO: 1 to SEQ ID NO: 43,177 and in SEQ ID NO: 43,220 to 43,288.

Advantageously, the invention relates to the vector as defined above, the vector further comprising:
a prokaryotic replication origin; or
a sequence coding for a protein allowing the resistant to an antibiotic, or both.

Advantageously, the vector as defined above may also contain a prokaryotic replication origin, in order to allow DNA replication in bacterial cells. It is also relevant to have a gene for the selection of the bacterial transformed cells, by using a gene coding for a protein allowing the resistance to an antibiotic, such as ampicillin, kanamycin, . . . .

In one advantageous embodiment, the vector described above is such that it comprises:
one of the mammalian genomic DNA replication origins comprising or consisting in one of the sequences as set forth in SEQ ID NO:1 to SEQ ID NO: 43177 and in SEQ ID NO: 43,220 to 43,288,
at least a sequence coding for a protein allowing the resistance or sensitivity to a compound specific to eukaryotic cells,
possibly an inducible transcription promoter able to promote transcription close or through the replication origin. and
a region independent to the mammalian genomic DNA replication origin allowing to insert a gene of interest and its expression.

The invention also relates to a vector comprising or consisting in a sequence acid sequence as set forth in SEQ ID NO: 43,290 to 43,358.

The invention relates also to a mammalian cell comprising a vector as defined above.

The mammal cells according to the invention contains a vector as defined above, i.e. a vector containing a mammalian replication origin. It is not necessary that this vector be inserted into the genome of the mammal host cell, since this vector contains a replication origin similar to the genomic DNA replication origin will replicate autonomously.

This vector will therefore be replicated as the genomic DNA does.

The invention also relates to a mammal, in particular a non-human mammal, comprising of cells as defined above.

The above animal, which preferably a non-human animal, such as a mouse, a rat, a monkey, a dog, a cat . . . contains at least one mammalian cell as defined above.

Advantageously, one or more organs of said animal may be colonized by the above-mentioned cells, i.e. some or all the cells of the organ contain a vector as defined above.

The invention also relates to the use of a vector as defined above, for expressing, preferably in vitro or ex vivo, in a mammalian cell, a gene of interest, the sequence of which being inserted in the vector in the region independent to the mammalian genomic DNA replication origin.

In this particular use, the gene of interest is placed under the control of a promoter, that allow its expression, and the expression of the corresponding protein.

By “the region independent to the mammalian genomic DNA replication origin”, it is meant in the invention that the gene of interest, is not cloned within the sequence of the origin, nor in the same multi cloning site. It could be therefore advantageous, in the above described vector, that an additional multicloning site be inserted in the vector, for the purpose of the cloning of the gene of interest.

The above vector can contain 2 or more mammalian genomic DNA replication origins, identical or different. Increasing the number of copy of mammalian genomic DNA replication origin will increase the replicative properties of the vector in mammal cells, as illustrated in the Examples.

The invention also relates to a computer program product implemented on an appropriated support comprising instructions to execute the steps b- to c- of the method as defined above.

The invention relates to software or a computer program product designed to implement the above-mentioned method and/or comprising portions/means/instructions of program code for executing said method when said program is executed on a computer. Advantageously, said program is provided on a data-recording support that can be read by a computer. Such a support is not limited to a portable recording support such as a CD-ROM but can also form part of a device comprising an internal memory of a computer (for example RAMs and/or ROMs), or of a device with external memory such as hard disks or USB sticks, or a proximity or remote server.

The computer program is adapted to carry out the step b and c of the above described method.

The invention will be better understood in the light of the following figure and the following example.

### LEGEND TO THE FIGURES

FIG. 1 shows Experimental workflow. SNS-seq was performed on three untransformed (hESC H9, patient derived hematopoietic cells (HC), and patient derived Human Mammary Epithelial Cells (HMEC), and 3 immortalized cell types (total n=19). Immortalized cells were obtained through a reduction of TP53 mRNA levels (ImM-1, p53^{KD}) or further expression of oncogenes RAS (ImM-2, +RAS) or WNT (ImM-3, +WNT) in HMEC cells.

FIG. 2: UCSC genome browser snapshots of the human replication origin (MYC origin) captured by SNS-seq. Representative SNS-seq read-profiles, published positions of ORC2- (red) and MCM7-bound (blue) regions and the GENCODE genes (v25) are shown. The position of origins defined in this study is shown on top; red: high-activity origins (core origins), light pink: low-activity origins (stochastic origins).

FIG. 3 represents a boxplot showing the average origin activity (normalized SNS-seq counts across all samples, in Log2) per each quantile (x-axis represents Q1-Q10 origins). Line within the boxplot represents median, whereas the bounds of the box define the first and third quartiles. Bottom and top of whiskers represent minimum and maximum numbers respectively for each boxplot.

FIG. 4: Q1 and Q2 origins host the overwhelming majority of initiation events in untransformed cell types. Pie chart representing the percentage of DNA replication initiation events (normalized SNS-seq counts) that originate from Q1, Q2 or Q3-10 origins in the indicated untransformed cell types.

FIG. 5 represents a Density plots showing the distribution of the distances to nearest origin (x-axis, in Kb) for core origins (left panel) and stochastic origins (right panel). In gray are control density plots that show the distribution of the distances between core/stochastic origins to the nearest randomized genomic region of the same size and number as origins. Both frequency plots were significantly different from randomized distributions (p≤2.2E-16, Chi-square Goodness-of-Fit test in R with observed and expected values for frequency).

FIG. 6 represents Pearson's correlation coefficient (r) of origin activities between cell types.

FIG. 7 represents Euler diagrams showing the fraction of core and stochastic origins shared by the untransformed cell types.

FIG. 8 represents Bar plots show the percentage of core origins that were identified as origin regions by another SNS-seq study (black), and the expected amount of overlap with control regions (white, dotted line). Control regions in this figure are regions of equal size to core origins that are located in randomized coordinates of the human genome. P-value obtained by Chi-square Goodness-of-Fit test.

FIG. 9 represents Bar plots representing the percentage of regions identified by INI-seq (in black) that overlap origins identified in this study. Dotted bar represents the expected amount of overlap with control regions. P-value obtained by Chi-square Goodness-of-Fit test.

FIG. 10 is the same figure as FIG. 9 for OK-seq regions.

FIG. 11 represents the percentage of core origins that overlap with pre-RC components ORC2 (within ±2 Kb; in red) and MCM7 (direct overlap, in blue). Dotted bars represent the expected amount of overlap with control regions. P-values obtained by Chi-square Goodness-of-Fit test.

FIG. 12 is the same figure as FIG. 11 for core origins found in clusters.

FIG. 13 represents Bar plots show the percentage of ORC1-(13,000) and ORC2-bound (55,000) sites that host DNA replication initiation within 2 Kb. Dotted bars represent overlap with control regions. P-values obtained by Chi-square Goodness-of-Fit test.

FIG. 14 is a schematic summary of origin activity in a single cell type.

FIG. 15 is a schematic summary of origin activity in the different cell types.

FIG. 16 represents Bar plots showing the percentage of all, hESC, hESC-specific, and Q1 human origins with homology to mouse (light green). Also indicated are regions in the human genome with a homologous region in the mouse (light green). Regions that are also origins in mouse are dark green. On the right, are bar plots showing the percentage of the corresponding shuffled genomic regions.

FIG. 17 represents cumulative Phastcon20 way scores plotted for human DNA replication initiation sites, similar-sized control regions (dotted), Refseq exons, promoters (defined as 500 bp upstream of TSS regions) and introns.

FIG. 18 represents a graph showing the percentage of origins in each quantile that overlap with G4 defined by G4Hunter (in silico) or mismatches (in vitro G4). Dotted lines (CTL) represent overlap with control regions.

FIG. 19 represents the base content of the regions flanking human DNA replication origins and control genomic regions. Frequency plots are centred at the origin summits. The base frequency represents the proportion of each base (0 to 1). The human genome is composed of 30% A, T and 20% G, C as indicated by genomic average. Origins are oriented with the highest G-content upstream.

FIG. 20 represents a Density plot representing the frequency of the distance measured between the initiation site summit (dotted line) and the centre/summit of the nearest ORC1 (red), ORC2 (dark red) and MCM7 (blue) bound regions. Origins are oriented with the highest G-content upstream.

FIG. 21 is the same figure as FIG. 20, but for stochastic origins.

FIG. 22 is a Schematic representation of a core origin. The vertical line represents the IS summit. The nearest ORC1, ORC2 and MCM7 peak centers are presented, as well as their average distance from the core IS summit. The average size of the ORC1, ORC2 and MCM7 binding sites is indicated on the left.

FIG. 23 represents a bar plot showing the percentage of origins that can be predicted based on the genome-scanning (GS) algorithm. Dotted bars represent the expected amount of overlap with control regions. The pie chart shows the percentage of false positive results (grey). P-values obtained by Chi-square Goodness-of-Fit test using observed and expected values for overlap.

FIG. 24 represents the Percentage of origins in each quantile predictable by the GS algorithm as in FIG. 23.

FIG. 25 represents the Percentage of Mus musculus origins predicted by the GS algorithm as in FIG. 23.

FIG. 26 represents Bar plots representing the percentage of core origins that can be predicted using a combination of GS algorithm and two different machine learning algorithms (single vector machine (SVM) and logistic regression (LR) with greedy feature selection). P-values obtained by Chi-square Goodness-of-Fit test using observed and expected values for overlap.

FIG. 27 is schema showing the properties of the regions predicted to be origins. G-richness in the immediate (0.5 Kb) and distal (2 Kb) upstream region to the initiation site are predictive parameters.

FIG. 28 represents a plot representing the percentage of DNA replication origins in each quantile that overlap a promoter region (±2 Kb of TSS) of a GENCODE gene (in red). Overlaps with control regions (paler color) which are randomly shuffled genomic regions of equal size and number as the origins are also shown. P-values obtained by Chi-square Goodness-of-Fit test using observed and expected values for overlap.

FIG. 29: As in FIG. 28 for overlaps with intergenic regions (>2 Kb upstream of a GENCODE gene, TSS are excluded).

FIG. 30: As in FIG. 28 for overlaps with gene body (genic region 2 Kb downstream of the TSS excluded).

FIG. 31 represents Bar plot representing percentage of CpG-containing gene promoters that host a DNA replication origin within +/−2 Kb of their TSS. Promoters with different transcriptional activity levels in hematopoietic cells are shown (silent=0, low=0-15, medium=15-60, high=>60 RPKM). In this figure, a promoter is considered CpG-containing (CpG(+)) if a CpG island is present within +/−2 Kb of the TSS (Gencode v25).

FIG. 32 represents Bar plot showing the average number of origins localized within 2 Kb of the TSS of genes with different transcriptional output levels (silent=0, low=0-15, medium=15-60, high=>60 RPKM) in hematopoietic cells.

FIG. 33 represents Boxplots showing the average activity of origins localized within 2 Kb of the TSS of genes with different transcriptional output levels as in (d) in hematopoietic cells. p-values were obtained using the Wilcoxon test in R.

FIG. 34 represents Dot plot shows the correlation of transcriptional output of CpGi(+) promoters in hematopoietic progenitors (y-axis; RPKMs, Log2) and the activity of core origins located within ±2 Kb of the TSS of these genes in hematopoietic progenitors (x-axis; normalized SNS-seq counts, Log2). Top and bottom 5% outliers were removed. The Pearson's correlation coefficient (r) and p-value for correlation is indicated on the top, and trendline is shown.

FIG. 35: As in FIG. 31 for CpGi(−) promoter regions.

FIG. 36: As in FIG. 32 for CpGi(−) promoter regions.

FIG. 37: As in FIG. 33 for CpGi(−) promoter regions.

FIG. 38: As in FIG. 34 for CpGi(−) promoter regions.

FIG. 39 represents a Schematic summary of findings. CpGi(+) promoters (black) tend to host DNA replication origins, irrespectively of their transcriptional status, while CpGi(−) promoters (grey) tend to host origins when they are transcriptionally active.

FIG. 40 represents a Euler diagrams showing the percentage of shared core and stochastic origins identified in untransformed (white) and immortalized (grey) cell lines.

FIG. 41: In immortalized cells stochastic origins are markedly increased. Bar plots showing the percentage of core and stochastic origins identified in each cell type.

FIG. 42 represents a Line plot showing the percentage of origins (Q1 to Q10) identified in immortalized and untransformed cells.

FIG. 43 represents the Percentage of origins in each quantile (untransformed Q1-10 in blue, immortalized Q1-Q10 in pink) that overlap with promoter regions (within +/−2 kb of the TSS). The expected chance overlap is shown with dotted lines (paler colors). P-values obtained by Chi-square Goodness-of-Fit test. P-value indicated in blue represent statistical analysis of overlaps in untransformed cells, while pink indicates immortalized cells.

FIG. 44: As in FIG. 43 for overlaps with gene body (excluding the TSS+2 kb region) of GENCODE (v25) genes.

FIG. 45: As in FIG. 43 for overlaps with regions enriched for heterochromatin-associated H3K9me3 histone mark (in hESC, left panel) and with regions defined as heterochromatin by HMM in hESC and K265 cells (right panel).

FIG. 46 represents Plot shows the core origin (red) density across topologically associating domains (TADs). Average origin density per bin (100 bins) across all TADs was plotted (y-axis, in origins/Mb). Core origin density is higher at the TAD borders, creating a “smiley” trend-line. p-values were obtained using the non-parametric Wilcoxon test in R.

FIG. 47: Same as in FIG. 46 but for stochastic origins.

FIG. 48 represents a Bar plot showing the sum of normalised mean SNS-seq signal (y-axis, total initiation) across 19 samples coming from both core and stochastic origins at TAD borders and TAD centers. The total amount of SNS-seq signal is 1.53 fold higher at TAD borders.

FIG. 49 represents the density of core origins active in HMEC (blue) and ImM-1 cells (orange) across TADs as in FIG. 46.

FIG. 50: Same as in FIG. 49 but for stochastic origins active in HMEC and ImM-1 cells.

FIG. 51: As in FIG. 48 for HMEC (parental) and immortalised ImM-1 cell types.

FIG. 52 represents a Summary of the experimental SNS-seq procedure with the appropriate controls.

FIG. 53 represents the origin activity heatmap of all the identified human origins in six different cell lines. Origins were sorted according to their average activity based on the number of normalized SNS-seq reads. Human origins were then divided in ten equal-size quantiles (Q1-Q10) that included 32,074 origins/each.

FIG. 54: Mappability is similar for origins across different quantiles. Percentage of origins in each quantile with at least 50% of the origin overlapping fully mappable regions (UCSC-Umap, mappability score of 1).

FIG. 55: Broad and diffuse initiation outside the mapped origin regions is not substantial. Analysis of total diffuse initiation in early and late replicating domains of the human genome reveals that only two cell types have some initiation signal outside origin regions. In hESC cells. 9.6% of all DNA replication initiation comes from early (but not late) replicating domains outside the identified origin regions. Im ImM-1 cell type, 14.7% of all initiation comes from late-replicating (but not early replicating) domains, outside the origin regions.

FIG. 56: Most core origins are clustered in the genome. Pie chart showing the percentage of core origins found (i) clustered (i.e., less than 7 kb from each other), (ii) loosely clustered (more than 7 kb, but less than 15 kb from each other), and (iii) isolated (more than 15 kb to the nearest core origin). Right panel depicts a schematic of the different clusters defined.

FIG. 57: A similar number of regions in the mouse genome also host the bulk of DNA replication initiation events. Pie chart showing the percentage of normalized SNS-seq tags that include the most active 64,148 origins (same number as in human cells) and the remaining lower activity origins.

FIG. 58 represents a Euler diagrams showing the fraction of origins shared by three immortalized cell lines.

FIG. 59 represents Black dots show the percentage of origins in each quantile that overlap origins detected in a previous SNS-seq study. Grey dots represent the expected chance overlaps of randomly shuffled, control genomic regions of equal size and number as our origins. P-values obtained by Chi-square Goodness-of-Fit test using observed and expected values for overlap.

FIG. 60: As in FIG. 59 for regions identified by INI-seq. Red dots depict the percentage of early-firing origins identified by INI-seq, which is an in vitro method that identifies earliest firing origins.

FIG. 61: As in FIG. 59 for OK-seq regions.

FIG. 62: Tightly clustered core origins are more likely to be identified by the alternative origin mapping method OK-seq. Bar plot showing the percentage of tightly clustered core origins (in black) that overlap with DNA replication initiation zones identified by OK-seq. Dotted bars represent the expected chance overlap of randomly shuffled, control genomic regions of equal size and number to OK-seq regions. P-values obtained by Chi-square Goodness-of-Fit test using observed and expected values for overlap.

FIG. 63: Core origins overlap with the pre-RC components ORC1 and ORC2 binding sites. Graph shows the percentage of origins in each quantile that overlap with regions bound by ORC1 or ORC2 (red) or ORC2 (blue) within ±2 kb. Paler coloured dots represent the expected chance overlap of randomly shuffled, control genomic regions of equal size and number as our origins.

FIG. 64: ORC2 binding sites that occupy larger genomic regions are more likely to be associated with DNA replication origins. Pie chart represents the percentage of ORC2-bound sites in the genome that intersect a core or a stochastic origin (within ±2 Kb). Left panel represents ORC2-bound regions longer than 1 Kb, and the right panel represents ORC2-bound regions longer than 2 Kb. p-values were obtained using the Chi-square of Goodness-of-Fit test in R with observed and expected overlap values.

FIG. 65: Same as in FIG. 64 for ORC1-bound regions.

FIG. 66: Core origins (Q1 and Q2) have conserved sequences upstream of the initiation site. Graph represents averaged Phastcon20scores of human origins (Q1-Q10), centered on the origin summit with flanking regions on each side. Origins are oriented to have the G-rich regions upstream.

FIG. 67: As depicted in FIG. 66 for origins that are associated or not associated with a TSS within +/−2 Kb.

FIG. 68 represents Bar plot representing the percentage of core and stochastic origins that overlap a putative G4 structure (in black) as defined by any one of the two methods used to define G4 structures (mismatch scoring or G4Hunter). Dotted lines represent expected overlaps with control regions, which are randomized regions of the genome of equal size and number to our origin regions. P-values represent Chi-square Goodness-of-Fit test using observed and expected values for overlap. (*) Please note that stochastic origins Q3-7 significantly overlap G4 regions (maximum p=0.0002) while Q8-10 do not.

FIG. 69: Motif enrichment analysis (using HOMER) for the regions covering 400 bp upstream of oriented core origins summits. Analysis in this figure represents enrichment over randomized genomic regions.

FIG. 70: Left panel represents motif enrichment over randomized genomic regions that contain the same C and G frequency as core origins. Right panel represents motif enrichment over randomized genomic regions that contain the same frequency of the dinucleotide “CG”.

FIG. 71 is a schematic diagram of the algorithm used to predict origins based on a DNA hyper-motif.

FIG. 72: Base content of the regions flanking mouse DNA replication (core and stochastic) origins and control genomic regions. Frequency plots are centred at the origin summits (highest point of the peak in a read pile-up). The base frequency represents the proportion of each base in sliding windows of 100 bp, on a scale from 0 to 1. Origins are oriented to have the side with the highest G-content upstream (see Methods for details).

FIG. 73: False positive rates (in gray) for three different machine learning algorithm methods. LR represents logistic regression with greedy feature selection, SVM represents univariate feature selection and single vector machine and uLR represents logistic regression with univariate feature selection.

FIG. 74: Different machine learning methods predict virtually the same core origins. Eulerr diagram (drawn to size) showing the overlap of core origins predicted by each machine learning method.

FIG. 75: The importance of each of the 22 features used for each machine learning algorithm. Top panel represents the weights assigned to each feature by the LR algorithm. Bottom panel represents the weights assigned to each feature by the SVM algorithm. The detailed explanation of each feature (x-axis) can be found in Table 2. Y-axis is of arbitrary units representing the importance assigned to each variable by each algorithm.

FIG. 76 represents a Bar plot representing percentage of all Gencode (v25) gene promoters that host a DNA replication origin within +/−2 Kb of their TSS. Promoters with different transcriptional activity levels in hematopoietic cells are shown (silent=0, low=0-15, medium=15-60, high=>60 RPKM).

FIG. 77 represents a Bar plot showing the average number of origins localized within the promoter region (+/−2 Kb of the TSS) of genes with different transcriptional output levels (silent=0, low=0-15, medium=15-60, high=>60 RPKM) in hematopoietic cells.

FIG. 78 represents Boxplots showing the average activity of origins localized in the promoter region (+/−2 Kb of the TSS) of genes with different transcriptional output levels as in (d) in hematopoietic cells. p-values were obtained using the Wilcoxon test in R. Line within the boxplot represents median, whereas the bounds of the box define the first and third quartiles. Bottom and top of whiskers represent minimum and maximum numbers respectively for each boxplot.

FIG. 79 is a Schematic summary of the hematopoietic cell (HC) differentiation protocol. HC (CD34+) were isolated from three independent human cord blood donors and expanded in three independent cultures for 6-7 days. Then, erythropoietin (+EPO) was added to the culture medium (Day 0) for 6 days, and cells were harvested at day 0, day 3 and day 6 for SNS-seq and RNA-seq analysis.

FIG. 80: Origins with increased activity after erythrocyte differentiation (day 6) are in genomic regions that host genes related to erythrocyte differentiation. The genomic coordinates of origins that were significantly upregulated upon EPO addition (day 0 vs day 6) were analysed with GREAT. GREAT analysis was performed on genomic coordinates of the origins that were significantly upregulated upon EPO treatment (day 0 vs day 6). Origin regions were associated with genes using the single-gene (SG) rule of GREAT. Only one category came up as statistically significant at Binomial p-value p<0.05, which was plotted here.

FIG. 81: Silent genes are less likely to contain a CpG island (CpGi) near their promoter region. Bar plots represent the fraction of GENCODE (v25) genes with different transcriptional activity levels in hematopoietic cells (defined as in FIG. 76) that contain (CpG(+), in black) or not (CpG(−), in white) a CpGi within their TSS region (±2 Kb)

FIG. 82 represents boxplots showing the average activity of origins localized within the promoter region (+/−2 Kb of the TSS) of genes with different transcriptional outputs (silent=0, low=0-15, medium=15-60, high=>60 RPKM). A G-rich TSS was defined as a TSS that contains a G-rich (>37% per 500 bp) stretch of DNA within ±2 Kb); p-values for significance in this figure are obtained using Wilcoxon test in R. Line within the boxplot represents median, whereas the bounds of the box define the first and third quartiles. Bottom and top of whiskers represent minimum and maximum numbers respectively for each boxplot.

FIG. 83 represents Pie charts representing the percentage of DNA replication initiation events (as assessed by normalized SNS-seq counts) at known origins that originate from Q1, Q2 (core origins) or Q3-10 (stochastic origins) in all cell types used in the invention.

FIG. 84: Origin G-rich sequence-specificity is lost upon immortalization. In immortalized cells, origins that are down-regulated (black bars) in comparison to the parental cell line (HMEC) tend to overlap with CpGi (left panel) or G4 (right panel) elements. In contrast, origins upregulated upon immortalization (in white bars) have less than expected overlaps with CpGi or G4 elements. For reference, the dotted line shows the percentage of all origins that overlap with a CpGi (left panels) or G4 (right panels) are shown.

FIG. 85: Same as in FIG. 84, but for core origins that are up- or down-regulated upon immortalization. For reference, the dotted line shows the percentage of core origins that overlap with a CpGi (left panels) or G4 (right panels) are shown.

FIG. 86: Mouse core (left panel) and stochastic (right panel) origin density across topologically associating domains (TADs) of mouse embryonic stem cells 6. Origin density along TAD domains (blue) or equal-size control regions (grey) was computed as follows. TADs were divided into 100 equal bins (slices) and the origin density in each bin was calculated as number of origins per Mb. The p-value was calculated using the non-parametric Wilcoxon test in R.

FIG. 87: Core origin density across TADs (determined in hESC H1) that are active in hESC H9 (left panel), HC (middle panel) or HMEC (right panel). Origin density along TADs was computed as in FIG. 86.

FIG. 88: Core origins coincide with putative regulatory elements. Plot shows the overlap of origins (Q1-Q10) with human genome regions that have putative regulatory functions (as defined by ReMap, >10 peaks).

FIG. 89: Principle of the DpnI test.

FIG. 90: pEPi-Del vector as a receptor vector for replication origins. The original vector is the pEPi vector. The pEPi-Del recipient vector was subcloned from pEPi by deleting the SV40 origin of replication.

FIG. 91: The pEPi-Del receptor vector was subcloned from pEPi by deleting the SV40 origin of replication. 293T (expressing T antigen) and 293 (without T antigen) cells were transfected with pEPi (SV40 origin) or pEPi-Del (lacking origin). At the end of the DpnI assay (FIG. 89), the number of colonies able to grow on Agar supplemented with kanamycin is estimated. Partial photos are shown.

FIG. 92: histograms showing the number of colonies in the experiment performed in 293T (left) or 293 (right).

FIG. 93: Controls to check the specificity of DpnI digestion. Presentation of the result of bacteria transformed with DpnI-digested plasmids prepared in either Dam (−) or Dam (+) bacteria.

FIG. 94: Histogram showing the percentage of replicated plasmids for each condition compared to the DpnI digestion specificity control.

FIG. 95: Evolution of the cloning strategy of the origins of interest.

FIG. 96: Reduction of the S/MAR sequence and replacement of the eGFP reporter gene by a gene allowing antibiotic selection of transfected cells.

FIG. 97: The reduction of the S/MAR sequence by MAR5 allows to maintain a good transfection efficiency after 2 days (left) and 5 days (right).

FIG. 98: The reduction of the S/MAR sequence by MAR5 preserves the replicative potential of the vector.

FIG. 99: Substitution of the eGFP reporter gene by the puromycin resistance gene.

FIG. 100: Substitution of the eGFP reporter gene with the puromycin resistance gene allows assessment of replication up to at least 13 days.

FIG. 101: Properties of sequences containing the origins of replication to be inserted into the pPuroDel-MAR5-MCS receptor vector.

FIG. 102: pPuroDel-MAR5-MCS and pPuroDel-MAR5-λORI-MCS.

FIG. 103: Application of the rapid replication assay based on DpnI digestion of non-replicated plasmids to assess the replication capacity of plasmids contained in the vectORI library (per pool of 5 plasmids).

FIG. 104: graph showing the results of the replication capacity of the plasmids (6 days after transfection), for pools A-F.

FIG. 105: Migration profile on agarose gel of isolated clones, undigested, digested with NotI/SacI or BamHI/SacI.

FIG. 106: Migration profile on agarose gel of clone 15_2, undigested or digested with two enzymes.

FIG. 107: Migration profile on agarose gel of double (DBL) plasmids or single plasmids.

FIG. 108: schematic representation of single and double plasmids.

FIG. 109: histogram showing the ratio of replication between double and single plasmids.

### EXAMPLES

### Example 1—Characterization of Human Origin

DNA replication initiates from multiple genomic locations called replication origins. In metazoa, DNA sequence elements involved in origin specification remain elusive. The inventors examined pluripotent, primary, differentiating, and immortalized human cells, and demonstrate that a class of origins, termed core origins, is shared by different cell types and host ˜80% of all DNA replication initiation events in any cell population. The inventors detect a shared G-rich DNA sequence signature that coincides with most core origins in both human and mouse genomes. Transcription and G-rich elements can independently associate with replication origin activity. Computational algorithms show that core origins can be predicted, based solely on DNA sequence patterns but not on consensus motifs. Inventors results demonstrate that, despite an attributed stochasticity, core origins are chosen from a limited pool of genomic regions. Immortalization through oncogenic gene expression, but not normal cellular differentiation results in increased stochastic firing from heterochromatin and decreased origin density at TAD borders.

Methods

Cell and Tissue Culture

H9 hESC cells (WA-09; Wicell) were obtained from ES Cell International (ESI, Singapore) and were maintained according to supplier's instructions, as described60. Briefly, undifferentiated hESC were grown on mitomycin C-treated (10 g/ml, Sigma) mouse embryonic fibroblasts (used at the cell density of 4-6×10⁴ cells/cm²) and in medium constituted by 80% Knock-Out DMEM, 20% Knock-Out Serum Replacement, 1% non-essential amino acids, 1 mM L-glutamine, 0.1 mM p-mercaptoethanol. At passaging, 8 ng/ml human bFGF (Millipore or Eurobio) was added to the medium. Peripheral blood mononuclear cells (referred to as hematopoietic cells, HC) were isolated from the umbilical cord blood of three independent human donors from the Clinique Saint Roch of Montpellier using the Ficoll density gradient method. HC were then purified by magnetic beads coupled with an anti-CD34 antibody, resulting in 0.5 to 1×10⁶ CD34+ cells, plated in culture and expanded ex vivo with supplemented Stem Span medium (IMDM+insulin, transferrin, BSA, 5% FCS+IL-3+IL6+SCF) for 6-7 days. Cell differentiation towards the erythropoietic lineage was induced by addition of erythropoietin (EPO, 3 units/mL). At different time points after EPO addition (day 0, 3 and 6), an aliquot of 50×10⁶ cells was collected and pelleted for molecular biology experiments (SNS-Seq, RNA-seq, RT-qPCRs for verification), while the remaining cells were left in culture. To verify erythropoietic differentiation, cells were phenotyped by flow cytometry analysis using antibodies against the hematopoietic/erythroid markers CD36, CD11b, GlyA, CD71, CD49d, CD34, CD98, IL3R, CD13 (Beckman Coulter). Differentiation into the erythrocyte linage upon EPO incubation was also confirmed by RT-qPCR analysis of RNA from cells at day 0, 3 and 6 using primers specific for linage markers.

HMEC cells were isolated and ImM1-3 cells were generated as previously described (available at https://www.biorxiv.org/content/early/2018/06/11/344465). Briefly, HMEC cells were initially immortalized using a stably transfected shRNA against TP53 (ImM-1). ImM-1 subclones were then generated by stable transfection of plasmids to over-express human RAS (ImM-2) or WNT (ImM-3).

Mouse ESC were cultured as previously described, and SNS-seq was carried2 on mESC (n=4) and neuronal progenitor cells (n=4). A total of 248,682 origins were identified and divided into 10 equal size quantiles as in human.

Ethical Permissions

All experiments, including those involving hESC and hematopoietic cells adhere to the guidelines established by the French Bioethics Laws, and the “Agence Frangaise de biomedicine”. CD34+ cells were isolated from umbilical cord blood obtained following delivery of deidentified full-term infants after written informed consent from the mothers. Use of these deidentified samples was determined to be exempt from ethical review by the University Hospital of Montpellier Institutional Review Board in accordance with the guidelines issued by the Office of Human Research Protections.

Nascent Strand Isolation (SNS-Seq) and Analysis

This method is the most precise procedure to map replication origins, although differences in SNS-seq and bioinformatics analysis methodologies, often using no or unsuitable controls, have affected the false-positive rate (FPR) in origin identification, resulting in varying properties attributed to metazoan origins. Here, the inventors are providing the inventors' SNS-seq protocol and an analysis pipeline. Briefly, cells were lysed with DNAzol, and then nascent strands were separated from genomic DNA based on sucrose gradient size fractionation. Fractions corresponding to 0.5-2 kb were pooled, incubated with T4 polynucleotide kinase (NEB) for 5′ end phosphorylation, and digested by overnight incubation with 140 units of A-exonuclease (Aexn). A second round of overnight digestion with 100 units of Aexn was performed. Aexn digests contaminating broken genomic DNA, but not RNA-primed nascent strands22. As experimental background control, high molecular weight genomic DNA for each cell type was heat-fragmented to the same size as nascent strands, incubated with RNase A/XRN-1 to remove the RNA primer in any contaminating nascent strand, and then treated with the same amounts of Aexn as the samples.

The inventors should stress that the conditions ours and most laboratories use for the SNS-Seq are strictly different from the report claiming a possible bias of the lambda exonuclease digestion. First, in classical SNS-Seq protocols, nascent RNA-primed at replication origins are purified by melting DNA followed by the separation of the nascent strands from the bulk parental DNA by sucrose gradient centrifugation. Only then, the purified nascent strands are digested with exhaustive lambda exonuclease digestion (more than 2 000 u/μg DNA). This is not the case in Foulk et al.62 in which bulk DNA is simply enriched in replication intermediates by using BND cellulose, which fractionates whole DNA that is partly single stranded. Lambda exonuclease is then used, resulting in an enzyme to DNA ratio 1000 to 3000 fold less than the ratio the inventors' laboratory employs. The inventors also repeatedly reported that all the inventors' control samples (Nascent strands from mitotic DNA, or G0 DNA, or high molecular weight DNA give very low enrichment values).

The quality of origin enrichment in each sample was first tested by qPCR using primers against known human replication origins. Primers used to detect origin activity for various origins are given in Table 4. Single stranded nascent strands were first purified using the CyScrib GFX Purification Kit (Illustra, 279606-02), then converted into double stranded DNA by random priming using DNA polymerase I (Klenow fragment) and the ArrayCGH Kit (Bioprime, 45-0048). cDNA libraries were prepared using the TrueSeq Chip Library Preparation Kit (Illumina). In parallel, heat-denatured genomic DNA input controls were also purified, random-primed and libraries prepared in the same manner. All samples were sequenced at the Montpellier GenomiX (MGX) facility using an Illumina HiSeq 2500 apparatus. bcl2fastq version 2.17 from Illumina was used to produce the fastq files. Illumina reads (50 bp, single-end) from each SNS-seq replicate were trimmed and aligned to hg38 using Bowtie2 (v2.2.6). Peaks were called using two peak calling programs: MACS264 (v2.2.1) and SICER65 (v1.1 modified to contain hg38 and mm10). Peaks were first called using MACS2 (default parameters plus—bw 500-p 1 e-5-s 60-m 10 30—gsize 2.7e9), followed by peak calling by SICER [parameters: redundancy threshold=1, window size (bp)=200, fragment size=150 effective genome fraction=0.85, gap size (bp)=600, FDR=le-3]. MACS2 peaks that intersect SICER peaks from each sample were merged using bedtools intersect to generate a comprehensive list of all human DNA initiation sites (IS) (Table 1). Blacklisted regions as defined by the ENCODE project (hg38, ENCSR636HFF) were subtracted from the final human DNA replication origin list. Mouse SNS-seq samples were processed as human SNS-seq and were also divided into quantiles (mQ1-mQ10) with each quantile containing 25,168 regions. Principal component and analysis and sample distances suggest that for cell types obtained from a single donor (i.e. HMEC), the overlap of origins is stronger amongst the replicates, than it is with other cell types. For donor-derived cell type (hematopoietic cells), the inventors observed that the SNS-seq samples are more similar within the same donor than with treatment status (i.e. treatment with EPO). This is in contrast with the RNA-seq data, where samples cluster according to their treatment (EPO) and not their origin (donor).

SNS-Seq Optimization and Quality Controls

Different experimental and bioinformatics methodologies have been used to obtain and analyse SNS-seq data. SNS-seq relies on the Aexn ability to specifically digest genomic DNA, while leaving the newly synthesized, RNA-primed nascent DNA intact. The inventors' analysis suggests that peak calling to define origin locations using 19 human SNS-seq samples in the absence of a background or experimental genomic DNA background identified approximately 200,000 and 150,000 peaks per sample respectively (mean number of peaks). This number is reduced by about half when an appropriate experimental background (heat-fragmented genomic DNA treated with RNAse and Aexn) is used, suggesting that the use of appropriate backgrounds is crucial to reduce false positives in peak-calling. When the inventors examined the nature of the background signal (RNAse+Aexn), the inventors observed only a minimal bias for G-rich regions (G4, G-rich, CG-rich) compared with randomized genomic regions (˜5 reads every 250 bp compared to ˜2 reads per 250 bp), a value insufficient to skew peak calling or the downstream analysis. This confirms that under the inventors' experimental conditions (in particular the inventors' λexn digestion conditions), putative G4, G- and GC-rich sequences are digested almost as efficiently as randomized DNA sequences, and that the background generated by regions resistant to digestion can be accounted for by using a suitable experimental background sample.

Summits and Orientation of Origins

Summits of origins were defined by calculating the highest number of SNS-seq reads in bins of 50 bp from 25 bp sliding windows, using bam files from all samples with a custom-made script (see code availability). Middle point of the bin with highest number of reads was considered the summit of the IS.

Origins were assigned a plus or a minus strand based on the G-content of the regions flanking the IS summit, such that the G-rich flanking region was oriented upstream (left) of the IS summit. To do this, the inventors calculated the number of G bases within 500 bp of each IS and assigned a (+) or a (−) strand to each origin to ensure that the 500 bp with the most number of G bases was oriented upstream of the IS.

Quantification, Classification, and Differential Activity of DNA Replication Origins

The bioinformatics on this project was supported by the high power computing cluster of University of Birmingham (CastLes and BlueBear). Quantification of the SNS-seq signal at DNA replication origins was done using the R-package DiffBind (v3.9, dba.sCore: TMM_minus_background), using all human/mouse origin coordinates. The TMM_minus command subtracted the background signal from the signal, before normalizing all 19 samples using a TMM based algorithm. “Normalized SNS-seq signal” in the manuscript refers to these values obtained after subtraction of background and TMM normalization. After the TMM normalization, the average normalized SNS-seq counts was calculated across the 19 samples for each origin and origins were ranked based on this value. Then, each origin was assigned to a quantile (Q1-Q10) that represents the origin position in the ranked list based on the average activity. For example, all origins in the top 10th percentile of activity were assigned to Q1, and all origins that ranked between the 10th and 20th percentile were in Q2, and so forth. Core origins were all Q1 and Q2 origins, while stochastic origins were in all the other quantiles (Q3 to Q10). Super origins were defined as having >50 normalized SNS-seq counts. Super origins were not included in the present analysis, but they are listed in Table 1, for readers interested in origins that are ultra-ubiquitous in the genome, such as the MYC and LaminB2 origins.

To determine the percentage of SNS-seq signal that falls in Core origins in each cell type, the total normalized (background-subtracted and normalized)SNS-seq signal and the fraction that belongs to Q1, Q2 and stochastic origins (Q3-Q10) were calculated.

Differential origin activity was calculated using the R libraries Diffbind (v3.9, TMM_minus) and DeSeq2 consecutively (see code availability for code).

Total initiation from early and late replicating domains

The early and late replicating domains were defined based on early and late replication domains common to H9 and CD34+ hematopoietic progenitors (Table 3). The origin coordinates (+/−2 kb) were removed (masked) from the domains. The SNS-seq signal was then quantified in these domains in both sample and background samples and normalised by RPKM. The signal was then calculated as: Total SNS-seq signal in sample over early replicating domains minus the Total SNS-seq signal in background over early replicating domains. The same was performed for late replicating domains. The average of 3 replicates was calculated for each cell type. For most cell types, the signal from non-origin replication domains did not exceed the background (i.e. was negative).

For hESC and IMM-1, where the inventors find that the initiation signal from early or late (respectively) replication domains exceeds the background, the inventors calculated the percentage of initiation from non-origin regions and origin regions and presented it in FIG. 55.

Clustering of Core Origins

Clustering of core origins was done using bedtools suite (v.2.25, command:bedtools cluster) with a maximal distance of 7 kb to the nearest core origin. Please note that bedtools does not perform categorical clustering. FIG. 62 shows a diagram for clustering. This means that 70% of core origins were found in clusters with at least 2 or more core origins that are at a maximal distance of 7 kb from another core origin. Isolated core origins, which make up 15% of core origins, are found more than 15 kb away from another core origin. The inventors also defined “loosely clustered” core origins, which were less than 15 kb but more than 7 kb to nearest core origin.

Comparison with OK-seq data: In order to define tightly clustered core origins, the inventors screened core origin clusters for those that contained 6 or more core origins. This produced 1039 clusters with an average size of 27,287 bp that contained 13,519 core origins. As OK-seq did not map X- and Y-chromosomes, the inventors also removed clusters mapping to these chromosomes for this comparison. The size of tight core origin clusters is comparable to the average initiation zone defined by OK-seq, which is −34 kb in size.

Distance Between IS and Pre-RC Components

Peak coordinates were downloaded from relevant sources (ORC124, ORC225 and MCM726) and mapped to hg38 version of the human genome. For ORC2 peaks, the inventors were provided with peak summits, while for ORC1 and MCM7 peaks peak centre was calculated as the peak summit. For overlaps with ORC1 and ORC2, peaks were extended +/−2 kb. In order to map the density of distance between Pre-RC components and IS summit, the inventors calculated the distance between the IS summit and the ORC2 summit or ORC1/MCM7 peak centre for all Pre-RC components within a distance of 10 kb of the IS. The inventors then plotted the density of these distances in R. As a control, this procedure was repeated with randomized genomic coordinates for pre-RC components, which did not show any enrichment upstream or downstream of IS.

Data Analysis and Plotting

Heatmaps, boxplots, and other plots were generated using ggplot2 (v3.1.0) and pheatmap (v1.0.12) in R. Pie charts were generated in Excel (v16.16.23) using data obtained in R. Both Pearson's and Spearman's correlation matrices were calculated in R using (command cor( ). Principal component analysis (PCA) and Euler diagrams were generated in R (command pca, library eulerr). Comparison between genomic coordinates (quantiles, alternative origin mapping methods, histone/Pre-RC binding sites) (intersectBed with a minimum overlap of 1 bp) as well as generation of randomized genomic coordinates were computed using the bedtools suite (bedtools shuffle-chrom, -noOverlapping, when possible). For computation of overlaps between ORC1 and ORC2 binding sites and origins, a maximum distance of 2 kb was taken as positive overlap. SNS-seq read density plots and heatmaps were generated using deeptools (plotProfile, plotHeatmap). When required, genome coordinates of different genome assemblies were converted using UCSC LiftOver (UCSC Toolkit). A full list of the genomic regions downloaded from external sources can be found in Table 3.

ReMap and Putative Enhancers

Origins were mapped onto the ReMap atlas55 (http://remap.cisreg.eu). ReMap results from an integrative analysis of transcriptional regulator ChIP-seq experiments from both Public and Encode datasets. The ReMap catalogue includes 80 million peaks from 485 transcription factors, transcription coactivators and chromatin-remodelling factors. Overlaps were assessed with bedtools (v.2.25), counting only regions with a minimum of 10 ChIP-seq peak overlap.

RNA-Seq and Analysis

RNA-seq profiling was performed on all HC samples in order to determine whether origin positions (SNS-Seq) are adapted with transcription programs (RNA-seq). To do so, ≥2 μg RNA was extracted and purified from an aliquot of 200 000 cells using TRIzol reagent (Sigma-Aldrich), followed by RNA purification using the RNEasy MiniKit (Qiagen 74104). RNA quality and quantity were analyzed using a Fragment Analyzer (Advanced Analytical). cDNA libraries were prepared by the Montpellier GenomiX facility using the TrueSeq Chip Library Preparation Kit (Illumina). After quality control (using FastQC v0.11.5), the TopHat software (version 2.1.1) was used for splice junction mapping through Bowtie2 (version 2.2.8) for mapping reads. Reads count on genes was performed using HTSeq-count (version 0.6.1p1). Gene annotations were downloaded from GENCODE, release 25 (GRCh38.p7, 23 Sep. 2016). Data were normalized by the relative log expression implemented in edgeR (version 3.8.6), and pairwise comparative statistical analysis to identify differential genes was performed using DeSeq2 (version 1.18.0 in R 3.2) (results were confirmed with edgeR version 3.8.6) using a generalized linear model.

Definition of G-Rich Regions (G4, CpGi, G-Rich)

Two methods were used to define G4 elements in the human genome based on (i) identification of mismatches induced by K+ and pyridostatin (PDS) treatment28 (in vitro G4) (ii) predictions by G4Hunter29 (in silico G4). Both datasets were generated in hg19, therefore the inventors have converted the inventors' origin coordinates to hg19 in order to examine overlaps.

CpG islands that were >300 bp in size were downloaded from UCSC (hg38). G-rich regions were defined as having a G density >37% within a 500 bp window in sliding windows of 100 bp (hg38) using bedtools commands bedtools makewindows, nuc and count. G-rich region list was used for the analysis in FIG. 79.

Analysis of base composition and motif discovery in genomic regions

Base composition was analysed using HOMER66, with 100 bp as window size taking the IS summit as the peak centre. The density data were visualized with Microsoft Excel. HOMER (v4.11.1) was used to search for motif enrichment in between the core origin summits and the 400 bp upstream regions (in oriented origins, this corresponds to the G-rich region). The inventors have used the following parameters; perl findMotifsGenome.pl hg38-size given-len 4,6,8,10,12-mask-norevopp [none, -noweight or -CpG].

Evolutionary Conservation Analysis

Refseq exons, introns and promoter regions (defined as −500 to 0 bp upstream of transcription start sites) and Phastcon scores (Phastcon20way) were downloaded from UCSC table browser (last update December 2017). Mean cumulative phastcon scores of each set of regions were calculated using R and bedtools suite (bedtools coverage). Human origin coordinates were converted to mouse coordinates either using LiftOver (UCSC toolkit) or BLAST. Very similar results were obtained with BLAST and LiftOver, the inventors presented the results from LiftOver.

Prediction of DNA Replication Origins in the Human and Mouse Genomes

The human and mouse genomes were divided into paired 500 bp windows (Watson and Crick strands separately) with a sliding window size of 100 bp using bedtools (makewindows) suite (˜30 Million windows for human genome). The number of each nucleotide (A,C,G,T) in each paired window was then calculated (bedtools nuc). Paired (consecutive) 500 bp windows were evaluated to fit a DNA sequence pattern (a hyper-motif) with minimum 28% G in the first window and minimum 25% G in the consecutive second window—and a requirement that G content drop by 8-40%, with a max A/T content 0.21 between the first and second window). This let us to identify 1,041,594 window pairs. The window pairs that were retained were then merged using bedtools merge to identify non-overlapping putative origin regions (228,442 regions with average size of 1.7 Kb).

Prediction of DNA Replication Origins in the Human and Mouse Genomes

Genome Scan Algorithm

The human and mouse genomes were divided into paired 500 bp windows (Watson and Crick strands separately) with a sliding window size of 100 bp using bedtools (makewindows) suite (˜30 Million windows for human genome, hg38). The number of each nucleotide (A,C,G,T) in each paired window was then calculated (bedtools nuc). Paired (consecutive) 500 bp windows were evaluated to fit a DNA sequence pattern (a hyper-motif) with minimum 28% G in the first window and minimum 25% G in the consecutive second window—and a requirement that G content drop by 8-40%, with a max A/T content 0.21 between the first and second window). The same algorithm was run for the reverse compliment strand (i.e. Crick strand, 28% C in second window, min 25% C in second window) on the same 30 M window pairs, bringing the number of window-pairs examined to 60 million.

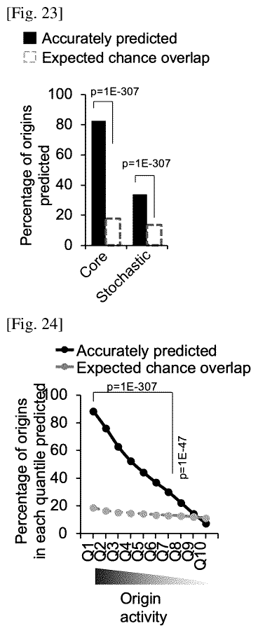

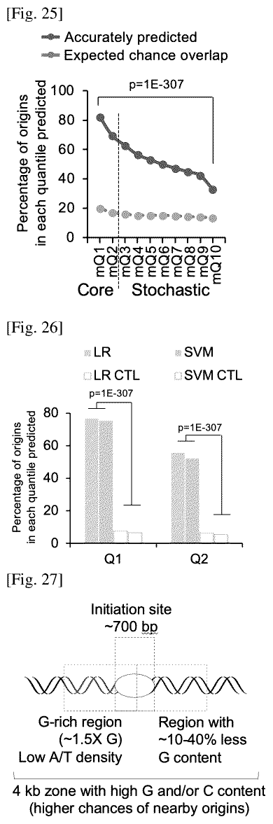

This let us to identify 1,041,594 window pairs. The window pairs that were retained were then merged using “bedtools merge” to identify non-overlapping putative origin regions (228,442 regions with average size of 1.7 Kb). This set of regions was used to define predictability of origins in FIGS. 23 and 24. For the mouse genome, the same algorithm was run with exactly the same parameters, which retains 689, 285 window pairs out of the (27×2 million possible pairs from mm10). Similarly, these regions were merged (bedtools merge) to generate 230,052 non-overlapping regions and intersected with mouse origins using bedtools (bedtools intersect -wa -u) to generate FIG. 25.

Machine Learning and Hyper-Motif Analysis

Predicted variable for the inventors' algorithm is the membership to the “origins” class defined by intersection of the non-overlapping coordinates with an origin (maximising the predictive power on core origins in particular).

30 million pairs of 500 bp windows were randomly split into two equally sized datasets. One of the datasets was reserved for the final validation at the end of the model development (test set). The other set was used for training and internal validation of the prediction model. Next, the training set was randomly split into 10 non-intersecting subsets and 10-fold internal cross-validation was performed (i.e. used 9 of these subsets for internal training and the remaining one for internal validation of the models, this was repeated 10 times, each time with a different validation subset). Initially, the Genome Scan algorithm was run on each one of those 10 internal training datasets. On the set of 1,041,594 regions generated by the GS algorithm (window pairs, see above), the inventors constructed a set of 22 parameters/predictors (see Tables 2) using domain knowledge. Then, machine learning procedures were applied to the output of the Genome Scan, thereby constructing a hierarchical classifier. This procedure was repeated 100 times for two different machine learning algorithms (i) logistic regression with greedy incremental feature and (ii) support vector machines with lasso regularisation. Greedy feature selection was performed by means of a modified version of statistical R-package CARRoT (Predicting Categorical and Continuous Outcomes Using One in Ten Rule, R CRAN package, 2018, Alina Bazarova and Marko Raseta, v1.0). The software was modified in such a way that would allow to incorporate merging of the output into non-intersecting genome regions by means of bedtools and then assessing the predictive power of the model given these regions. The support vector machine prediction was performed using R-package sparseSVM67 and additional scripting described above.

The inventors chose the models aiming at maximising their balanced (average class-wise) accuracy defined as 0.5*[TP/(TP+FN)+TN/(TN+FP)], where TP, TN, FP, FN stand for True Positives, True Negatives, False Positives, False Negatives. Due to the absence of the synthetically constructed negative instances of the origins these quantities were computed in terms of the overall length of the regions corresponding to true positive, true negative, false positive and false negative hits of 500 bp window pairs. The inventors kept on adding features to the greedy feature selection until improvement in predictive power was lower than 10\hat{ }-3. When working with SVM the inventors chose penalising parameters which led to the highest cross-validated predictive power as defined above. At the end of the procedure the inventors obtained 100 predictive models for each method which exhibited the highest predictive power for a given 10-fold cross-validation partition. For logistic regression, the best model emerged with the highest frequency of the predictors constituted by the features: UP_C_fraction, UP_G_fraction, Down_T_fraction, G_content_2 kb, rampG, AAA, GG, TTT (Tables 2). Once the training was complete, the chosen models based on 10-fold cross-validation were fitted with the whole original training set of 15 million pairs of 500 bp windows. The resulting trained models were then tested on the final hold-out test set (isolated from the training one in the very beginning and never touched throughout the model construction phase). Please note that each algorithm reported non-duplicate window pairs (i.e. if a window pair is retained with both forward and reverse scanning procedure by the genome scan algorithm, this window pair is reported once as positive by either machine learning algorithm).

In order to generate the predictions genome-wide, the trained model was run on the entire set of regions from GS resulting in 333,986 window pairs for LR and 279,195 window pairs for SVM called as positives by each algorithm. These window pairs were merged using bedtools (bedtools merge) to generate non-overlapping windows of 67,297 (LR) and 57,339 (SVM) regions. Please note that due to the sliding window pattern the inventors used to scan the genome, each window overlays 9 other windows, thus the same genomic regions are reported numerous times. The inventors remove the repeating regions by merging them, using bedtools merge, thus obtaining non-overlapping regions of the genome. These non-overlapping regions were used to generate the final predicted regions (i.e. FIG. 26 for core origins) or total false positive rate (regions not intersecting an origin, FIG. 73, normalised to average fragment length).

Calculation of Origin Density and Total Initiation Signal Across TAD Domains

To calculate the origin density across TAD domains, each TAD was divided into 100 bins (bedtools makewindows −n 100). As the bin size in each TAD was a fraction of the TAD size, the number of origins in each bin of the TAD was normalized to the bin size. To determine whether origin density across the TAD was significantly different in different cell types, the origin density across TADs for each bin was normalized to the 20 bins in the middle of each TAD (bin numbers 40-60). These values represent the differential origin density between the TAD middle and borders, rather than the overall origin density across the TAD.

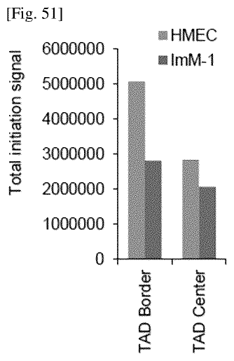

The inventors have calculated the sum of normalized (background subtracted) signal from origin regions that fall onto TAD borders or TAD centres (dataset on Table 3, FIGS. 48 and 51). As before, TAD domains were divided into 100 bins and the 20 bins (1-10,91-100) were defined as borders, while 20 bins (41-60) were considered as centers.

Statistical Significance

Different statistical tests were used depending on the data nature, as indicated in the figure legends. Specifically, the R commands “wilcoxon.test”, “t.test”, and “chisq.test” were used to measure statistical significance. p=1 E-307 and p=2E-16 represent the lowest value stored in the memory of R (depending on the version). The Chi.square test is essentially a one-sided test, while Wilcoxon assumes a non-parametric distribution.

Data Availability

Data downloaded from external sources can be found in Table 3. Raw read files for SNS-seq/RNA-seq and processed files can be found at the NCBI Gene Expression Omnibus (GEO) under the accession code GSE128477.

Code Availability

Scripts and other bioinformatics pipelines used to analyse SNS-seq data can be found at https://github.com/iakerman/SNS-seq.

Results

The landscape of DNA replication origins in the human genome

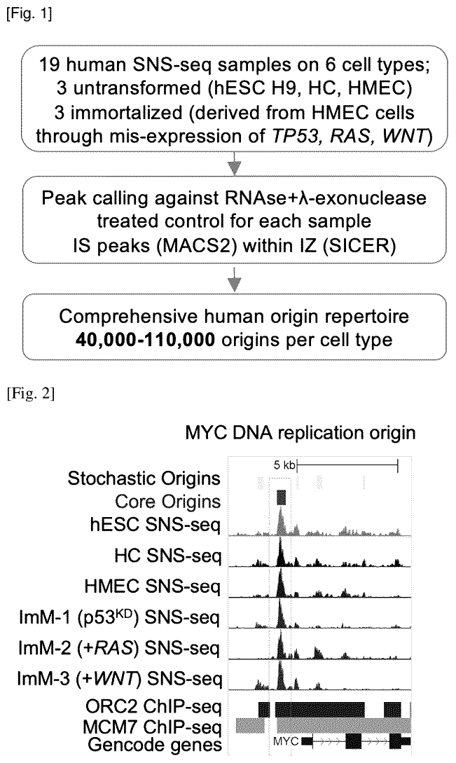

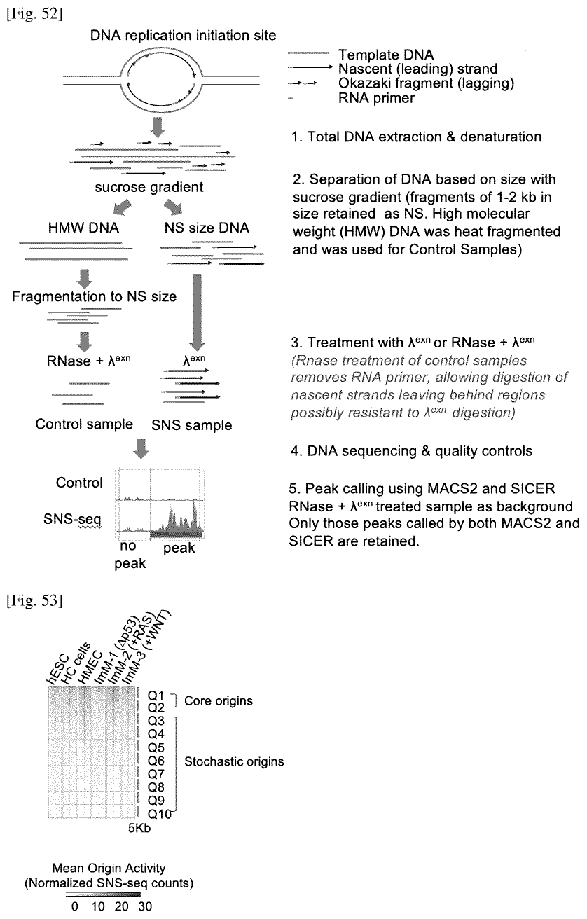

Using an optimized SNS-seq protocol (see Methods and FIG. 52), the inventors identified DNA replication IS from 19 human cell samples, representing three untransformed (human embryonic stem cells, hESC; cord blood CD34(+) hematopoietic cells, HC; primary human mammary epithelial cells, HMEC) and three immortalized cell types derived from the HMEC line (ImM-1, ImM-2, ImM-3) (FIG. 1). Owing to the high number of cell samples investigated, a total of 320,748 IS were identified, the overwhelming majority of which were low activity IS belonging to immortalized cell types (Table 1a, see following section). The IS repertoire included the previously identified human LaminB2, MYC, MCM4 and HSP70 origins (FIG. 2 and Table 1 b).

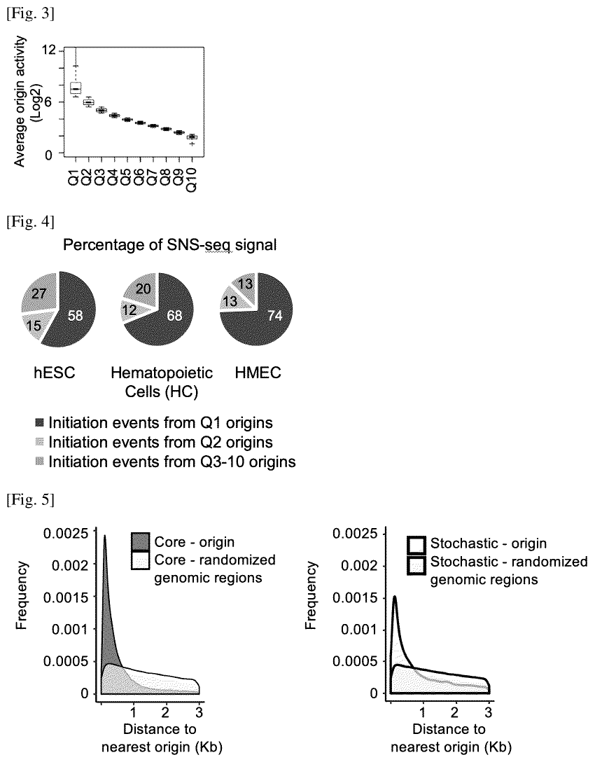

As the raw data clearly exhibited variations in replication origin activity, the inventors classified origins in ten quantiles, based on their average activity (i.e., mean normalized SNS-seq signal): from quantile 1 (Q1) that contained the top 10% (highest average activity) to quantile 10 (Q10) that included the bottom 10% (lowest average activity) of origins (FIG. 3, FIG. 53). Origins in each quantile displayed similar mappability, which is a measure of the ability of SNS-seq reads to be matched to the human genome. Therefore, the variation in SNS-seq signal at origins belonging to different quantiles were not due to the technical differences in the inventors' ability to map them (FIG. 54)

Strikingly, the inventors' classification revealed that 70-85% of the origin SNS-seq signal originated from Q1 and Q2 origins in all cell types analysed (FIG. 4, Table 1a). In addition, the inventors observe that almost all the enrichment of the SNS-seq signal across the genome comes from regions that are defined as origins in the inventors' study, suggesting that broad and diffuse initiation outside origin regions is not substantial (FIG. 55, see Methods). As the SNS-seq signal represents the amount of DNA replication initiation events that take place in a cell population, the inventors concluded that Q1 and Q2 origins host the majority of the initiation events, highlighting these 64,148 regions, termed “core origins”, as replication initiation hotspots, irrespective of the cell type.

The remaining 80% of IS (Q3-Q10, 256,600 regions), hereby termed “stochastic origins”, had low mean activity across 19 samples and only hosted −15-30% of total

SNS-seq signal in each cell type (FIG. 4, Table 1a).

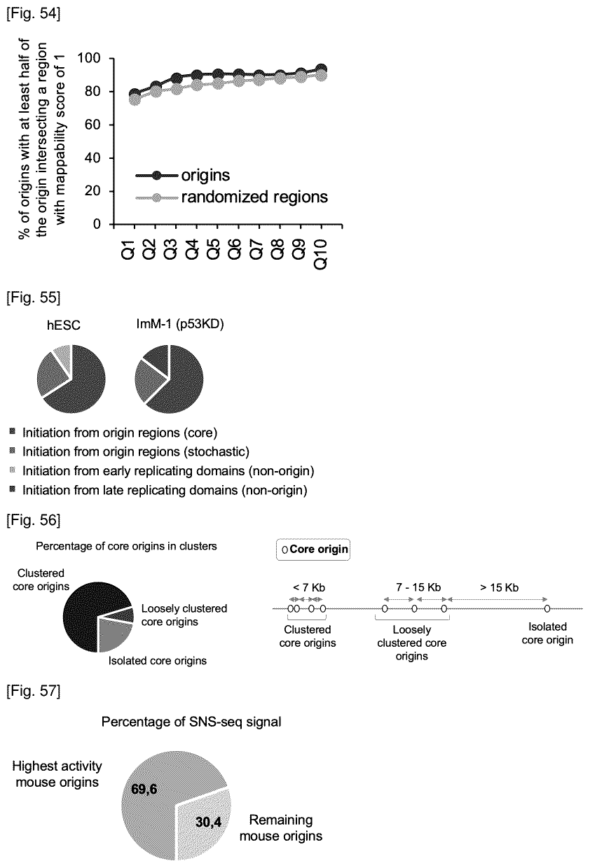

Most core origins were clustered together, because the distance to the nearest origin was shorter for core origins compared with stochastic origins or random distribution (FIG. 5, FIGS. 53 and 56). This is consistent with a previously observed community effect whereby clustered origins have higher activity than isolated origins4,10,22 (FIG. 56). Remarkably, a similar number of core origins in Mus musculus host 69% of all initiation events detectable by SNS-seq, suggesting that the core origins are a feature not specific to the human genome (FIG. 57).

The Position of Core Origins is Consistent

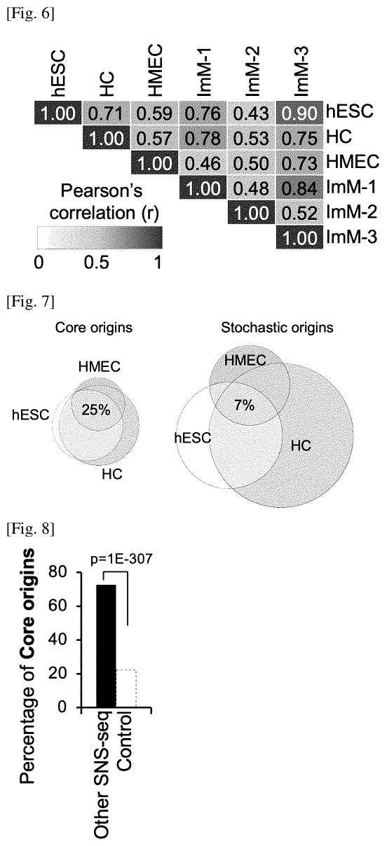

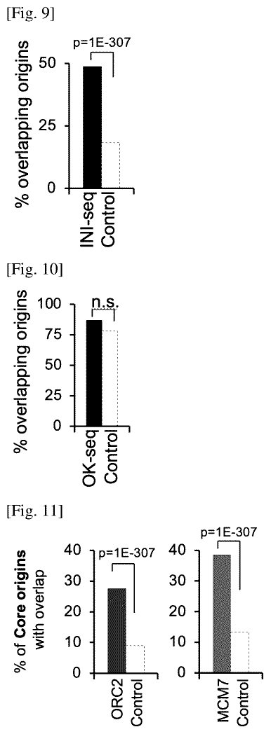

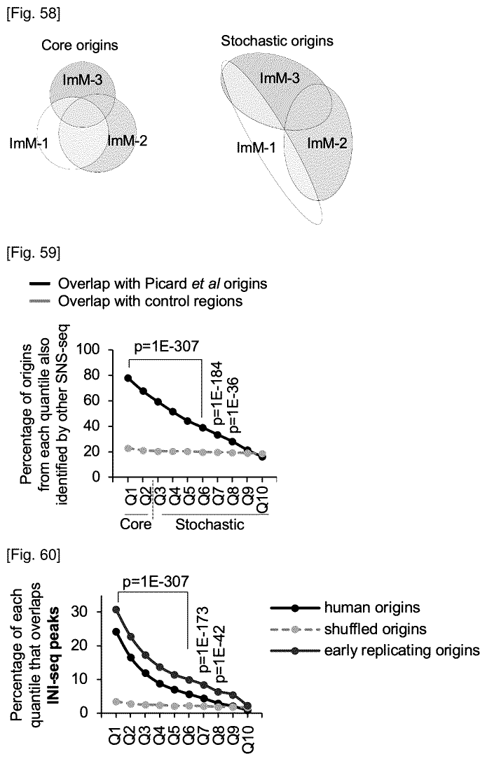

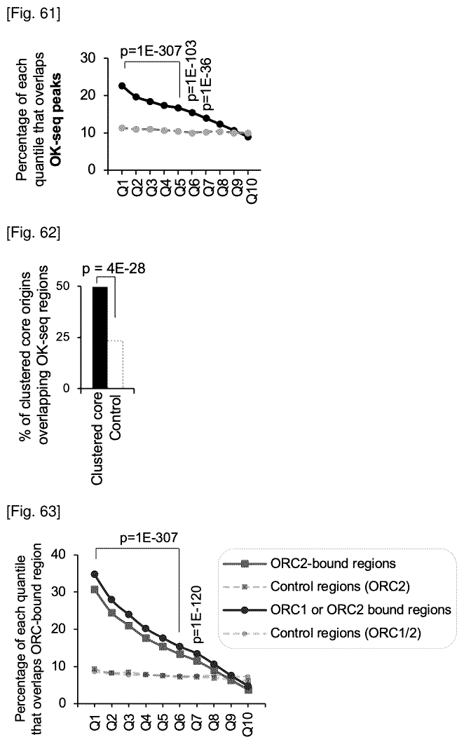

Origin activity was highly correlated in the different cell types (FIG. 6, average Pearson's r=0.69, p-value <2E-16 for all comparisons), suggesting that a given origin has similar levels of initiation in different cell types. About 77% of origins shared by the different cell types were core origins (Table 1a). Conversely, stochastic origins were less shared (FIG. 7, FIG. 58). In support of the inventors' findings that core origins are more ubiquitously active in different cell types, 72% of core origins were identified by an independent SNS-seq study using different cell types (FIG. 8, FIG. 59). Moreover, 49% of regions identified by a different origin mapping method (INI-seq) in a different cell line overlapped the inventors' origins, majority of which were core origins (FIG. 9). Early firing core origins were more likely to be identified by INI-seq, which maps early-firing origins (FIG. 60). In addition, almost all (87%) regions identified by OK-seq, overlapped origins identified in this study (FIG. 10). However, as this method only maps 5000 to 10 000 regions, with an average size of 34 kb; this overlap was not statistically significant. Nevertheless, core origins and core origins found in tight clusters (see Methods), which resemble initiation zones similar in size to those identified by OK-seq, overlapped significantly with regions identified by OK-seq (49.7%, FIGS. 61 and 62).

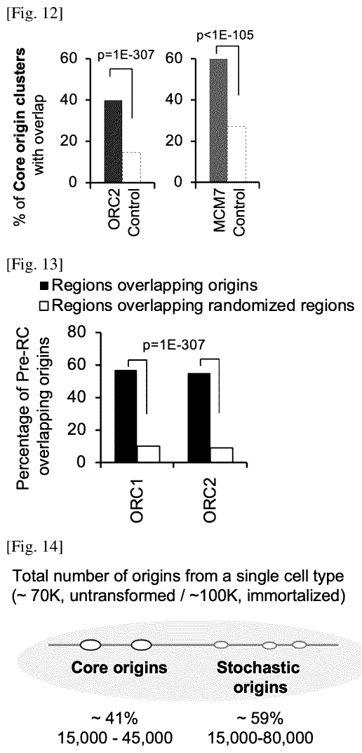

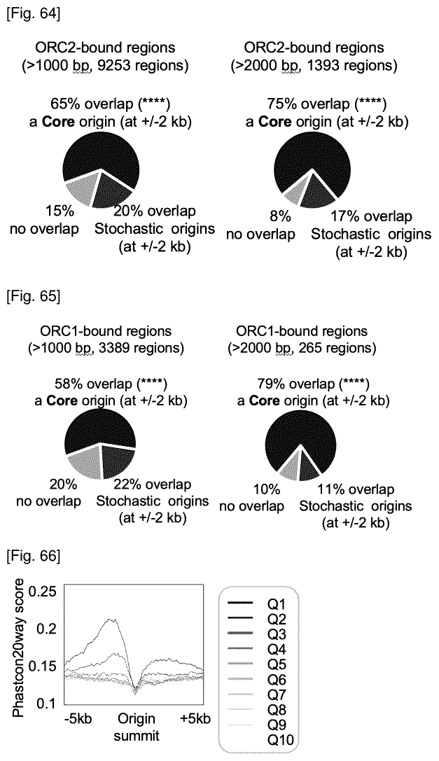

Core origins also coincided with regions previously shown to be bound by the pre-replication complex (pre-RC) components ORC1, ORC2 and MCM7. Specifically, 28% and 39% of core origins overlapped with ORC2 or MCM7 bound regions (FIG. 11, FIG. 63). Clustered core origins (initiation zones) overlapped with pre-RC component-bound regions more often (40% with ORC2 and 60% with MCM7, FIG. 12). Given that only about half of all core origins is active in any one cell type, the amount of overlap is suggestive that most active core origins are associated with pre-RC components ORC2 and MCM7. Reciprocally, 57% of ORC1- and 55% of ORC2-bound regions overlapped at least with one origin identified by SNS-seq (FIG. 13). Broader ORC1- or ORC2-bound regions, which might represent regions with multiple ORC1/2 binding events as suggested in S. pombe, were more likely to host an origin, and mostly a core origin (FIGS. 64 and 65).

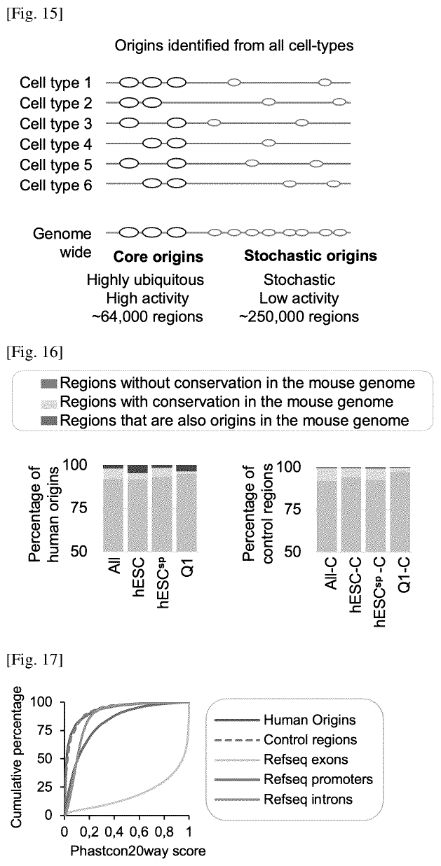

In summary, the inventors' analysis identified core origins that represent bona fide IS in different cell types, which are also identified by alternative origin mapping methods. On average, core origins represent ˜40% of all origins identified in a single cell type, representing on average ˜30,000 regions (FIGS. 14 and 15). It is worth noting that core origins are different from “constitutive/common origins” previously observed with SNS-seq data. The inventors' analysis has the highest number of samples amongst these studies and based on the inventors' data, the inventors infrequently observe origins that are active in every sample.

Human and Mouse Genomes Share a G-Rich Sequence Signature

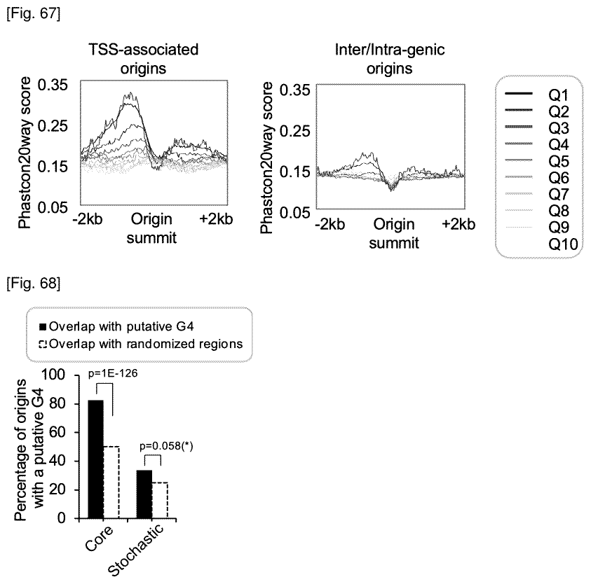

The inventors next investigated whether DNA replication initiation sites are placed in homologous regions across mouse and human genomes. The inventors find that only a small fraction (8%) of human origins have homologous regions in the mouse genome and only 2% are also identified as origins in mouse cells (FIG. 16, left panel). The inventors find a comparable level of homology for randomized genomic regions (7% conserved, 0.8% overlapping mouse origins, FIG. 16, right panel) suggesting that the majority of DNA replication initiation sites are not located in homologous regions in the mouse and human genomes. In accordance, the inventors observed a low level of sequence conservation of the origin DNA sequence compared to promoters and exonic regions across 20 mammalian species, reinforcing the idea that these sequences have appeared independently in the different lineages during evolution (FIG. 17). Interestingly, Phascon20way scores of regions flanking the origins (+/−5 Kb of origin summits), display moderately conserved regions 0.5-3 Kb upstream of the IS region for core origins, which are mostly attributable to regulatory elements/exonic sequences (FIGS. 66 and 67).

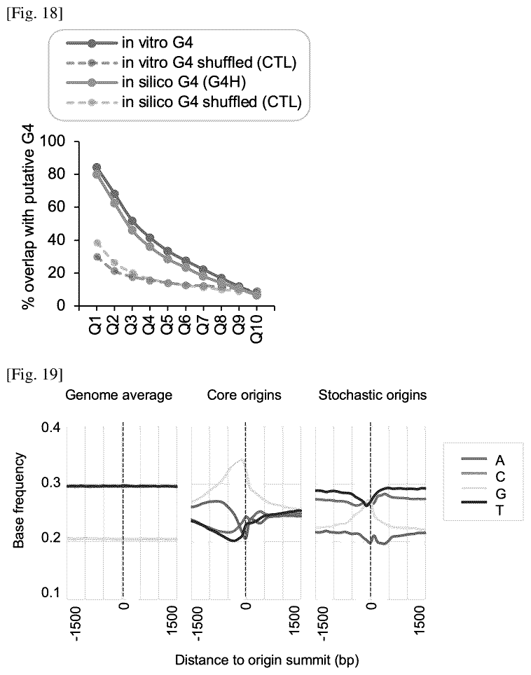

Despite lacking sequence homology, functional regions of the genome may contain sequence elements that are shared between species. Thus, the inventors next examined sequence elements that might be shared across replication origins of different species. To identify DNA sequence elements that coincide with origins, the inventors examined the relationship between the IS and G-rich putative G4 structures, which are helical DNA configurations that contain one or more guanine tetrads. 83% of core and 34% of stochastic origins contained at least one putative G4 element defined by two different methods (FIG. 18, FIG. 68). A large number of putative G4 elements has been predicted in human and mouse genomes, but as previously noted, only a fraction of them hosts an origin. Hence, the presence of a putative G4 element is not, on its own, a strong predictor of origin placement, but most core origins indeed contain a G4 element.

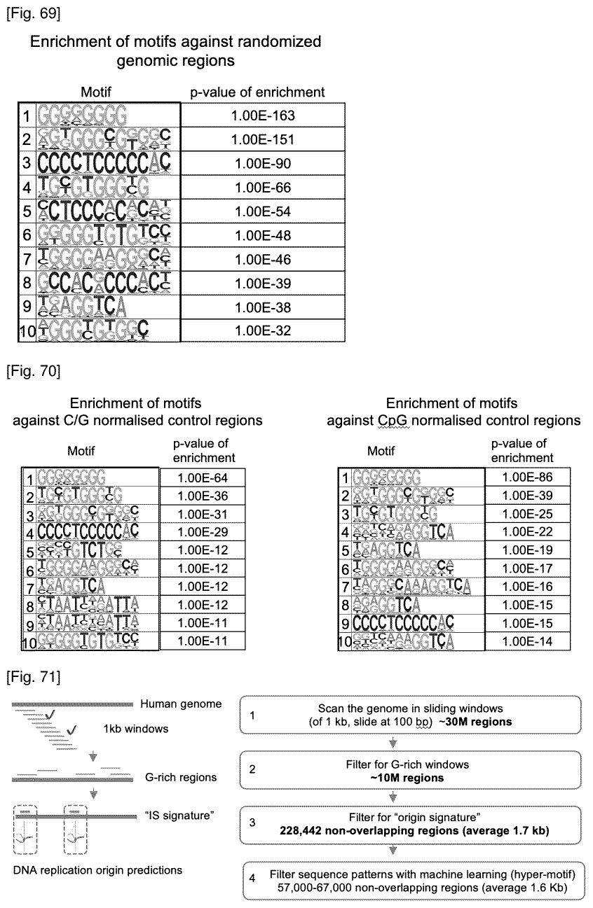

Similar to previous findings in mouse, a number of G-rich motifs upstream of the IS were evident (FIG. 69) and were enriched in origin sequences even after C/G and CpG content normalisation of the control regions (FIG. 70). Analysis of the base composition of human origins within ±1.5 Kb of the oriented IS summit confirmed that core origins were enriched in G-rich sequences with an asymmetrical enrichment up to 1.5 Kb upstream of the IS centre (FIG. 19).

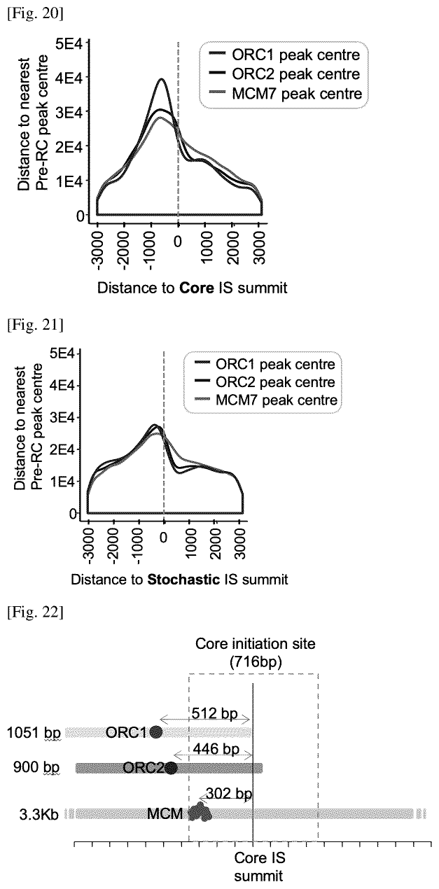

The inventors further asked how the replication origins determined in this study position relative to the placement of pre-RC factors on the genome. When the inventors aligned the positions of the pre-RC components ORC1, ORC2 and MCM7 relative to the IS, the inventors found that they were preferentially positioned upstream of the IS, near the G-rich region in both core and stochastic origins (FIGS. 20 and 21). In addition, the distances between the IS and these pre-RC factors recapitulated independent biochemical methods measuring positioning of pre-RC factor binding sites, such that the median distances between core IS (peak summit) and ORC1, ORC2 and MCM7 binding sites (peak centre) were 512, 446 and 302 bp, respectively. This positioned the peak of MCM complex downstream of the ORC subunits, at 300 bp from the IS (FIG. 22). Indeed, the MCM complex sits on at least 68 bp and binds to a neighboring nucleosome, increasing the size of the protected DNA up to 210 bp. In addition, the MCM helicase must unwind the DNA over a minimum length in order to allow the DNA polymerase to bind to the unwound DNA. The inventors believe that this result, linking the IS determined by SNS-seq and pre-RC binding sites determined by ChIP-seq, is a clear independent demonstration that the SNS-seq method accurately maps the initiation sites of DNA replication. Furthermore, the inventors' results show that the relative in vivo positioning of Pre-RC components and IS are similar to those determined by biochemical methods.

Origin Positioning can be Predicted Based on DNA Sequence

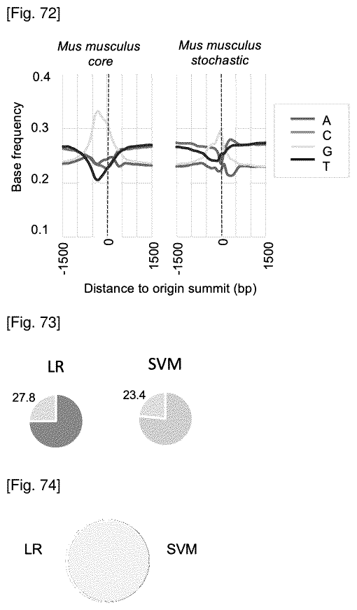

As strong origins display a G-rich profile (a putative sequence signature), the inventors asked whether DNA replication origins could be predicted from the DNA sequence alone. Classical motif search algorithms are designed to detect enrichment of short, but highly similar stretches of DNA, typically bound by transcription factors. Given the core origin size (average 716 bp), the inventors hypothesized that they may be specified by hyper-motifs, which are discriminatory DNA sequence patterns that are typically longer than classical transcription factor binding sites. To do this, the inventors modelled the asymmetrical base composition of the core origin and its flanking sequences and scanned the human genome for similar DNA sequence patterns (FIG. 71, see Methods). The genome scanning (GS) algorithm identified 228,442 non-overlapping regions which located 83% of core origins and 33% of stochastic origins with FPR of 66% (FIG. 23). The predictive ability of the GS algorithm decreased in parallel with the mean origin activity, suggesting that origins with higher activity (core) are more likely to contain discernible G-rich sequence elements (FIG. 24). The inventors' GS algorithm also predicted 76% of core and 54% of all origins in the mouse genome (FIG. 25), which display a similar G-rich sequence signature at core origins (FIG. 72). Asymmetrical base composition at origin sequences has previously been observed. Interestingly however, only the modelling of core origins, but not of stochastic or previously published origins led to high predictive power with the GS algorithm (see Methods). In conclusion, despite lack of evolutionary sequence conservation of DNA replication origins in these two mammalian species (FIGS. 16 and 17), the inventors' data suggests that most human and mouse core DNA replication origin positions can be predicted using DNA sequence alone based on the same G-rich DNA hyper-motif, suggesting that a conserved mechanism(s) governs origin selection in these vertebrate species.

To improve the predictive power and reduce FPR, the inventors modelled the DNA sequences around the predicted regions and used two different machine-learning (ML) algorithms (see Methods) to better differentiate true origins in the inventors' predictions. Modelling of the DNA sequences included using information, such as the density of di-, tri- and multi-nucleotides (CC, CG, GG, CGCG, etc.), inter-prediction distances, and the base composition variations (A, T, G, and C) of the DNA across a 4 kb region (see Methods). Remarkably, GS algorithm coupled with a ML algorithm (logistic regression with greedy feature selection, LR) identified 67,297 non-overlapping regions and predicted 67% of core origins with a total FPR 27.8% (FIG. 26, FIG. 73). In other words, a large proportion (67%) of core origins contain discernible DNA sequence patterns, and when these patterns are present in the genome, they are associated with an origin 72.2% of the time, in at least one cell type. Importantly, when the inventors employed a completely independent ML approach (SVM), this resulted in vastly overlapping predictions (FIG. 26, FIG. 74) with an FPR of 23.4% (FIG. 73). Coupling of GS and ML algorithms thus allowed the prediction of origin positions in a genome as large as the human genome.

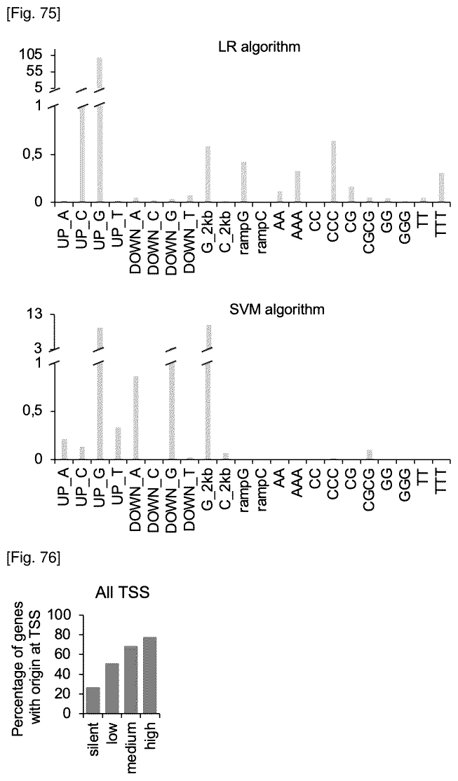

Both SVM and LR approaches identified the upstream G density as critical parameters for predictions (FIG. 27, FIG. 75). This is in accordance with the presence of an origin G-rich Repeated Element (OGRE) or tandemly arranged multiple (up to 6-12) G4 structures as well as ultra-short C/G-rich nucleotide motifs found at human, mouse and chicken origins.

Cell Differentiation Alters Origin Positioning and Activity

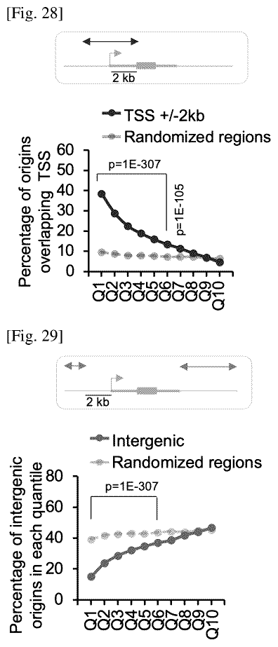

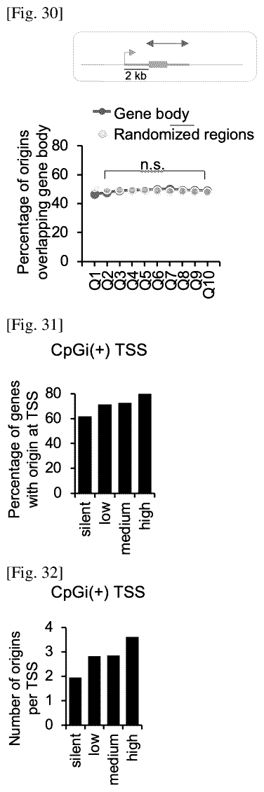

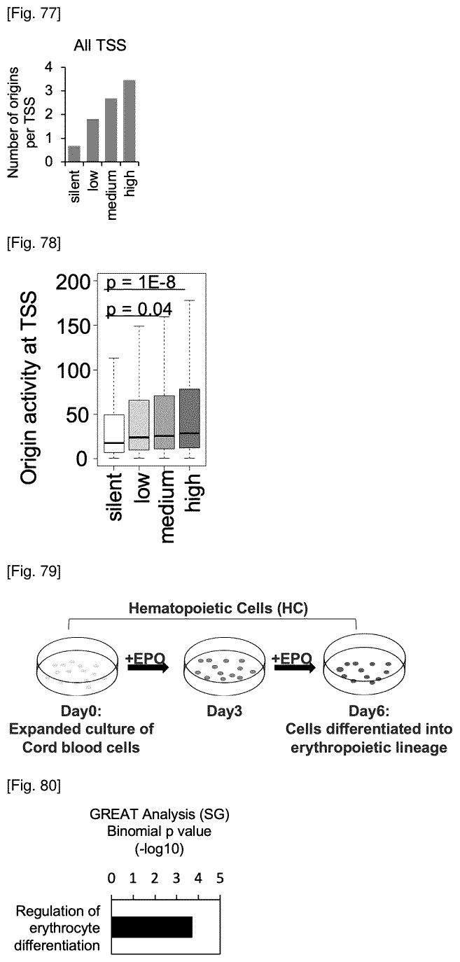

The inventors observed that in the human genome, core origins were preferentially placed near promoter regions and depleted from intergenic regions (FIGS. 28, 29 and 30). This is in agreement with a number of studies suggested that transcription is a predictive factor for DNA replication origin specification with varying degrees of correlation. The inventors' data also suggests that in hematopoietic cells, genes with higher transcriptional activity were more likely to host an origin in their promoter region (FIG. 76). Both the number and activity of origins within promoter regions increased with the promoter transcriptional output (FIGS. 77 and 78). Either RNA synthesis activity per se, or open chromatin induced by transcription complex assembly might favor pre-RC formation. However, the correlation between the position of core origins at promoter and intergenic regions (FIGS. 28 and 29) is not observed for gene bodies (FIG. 30). This finding suggests an impact of the chromatin environment of the promoter, rather than RNA synthesis per se, in the preferential localization of origins at promoter regions.

The inventors next used hematopoietic cells undergoing erythropoiesis to examine the impact of changing transcriptional landscape on origin specification. CD34(+) hematopoietic cells were isolated from human cord blood and differentiated towards erythropoietic linage using erythropoietin (EPO) (FIG. 79). Gene ontology analysis (GREAT) revealed a single enriched set of genes with origins activity increased upon erythrocyte differentiation (FIG. 80) suggesting that DNA replication origins are recruited to gene domains undergoing transcriptional and epigenetic changes.

G-Rich and Transcription Impact on Origin Activity

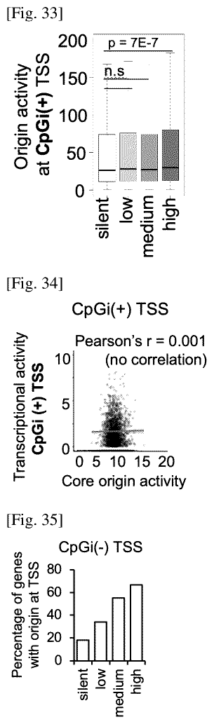

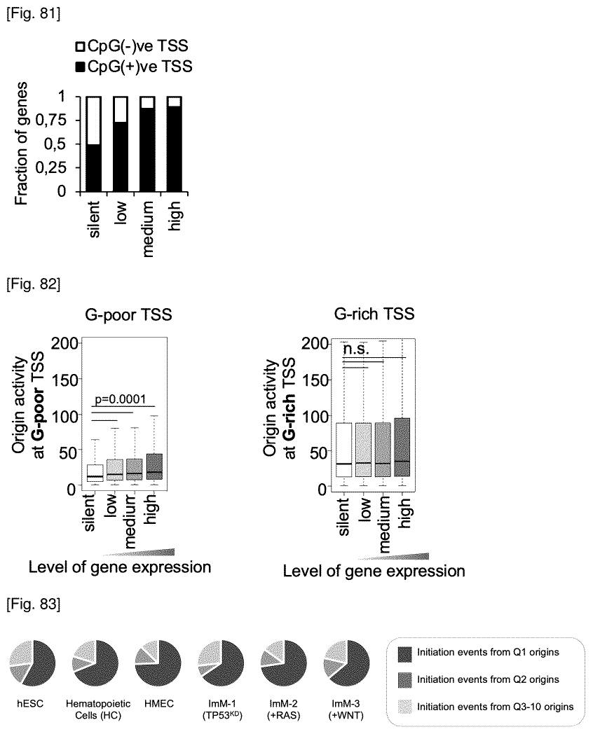

In HCs, 89% of highly expressed genes hosted a CpGi (a G-rich region) in their promoter, whereas only 48% of silent gene promoters hosted CpGi (FIG. 81). Therefore, the inventors asked whether the concomitant presence of a CpGi (or a G-rich stretch) and high transcription activity was required for high origin activity in hematopoietic cells. The inventors did not observe a profound impact of transcription on origin numbers, clustering or activity near CpGi(+) promoters (FIGS. 31, 32 and 33). In addition, DNA replication initiation activity from CpGi(+) TSS did not correlate with transcriptional activity (Pearson's r<0.01, FIG. 34).

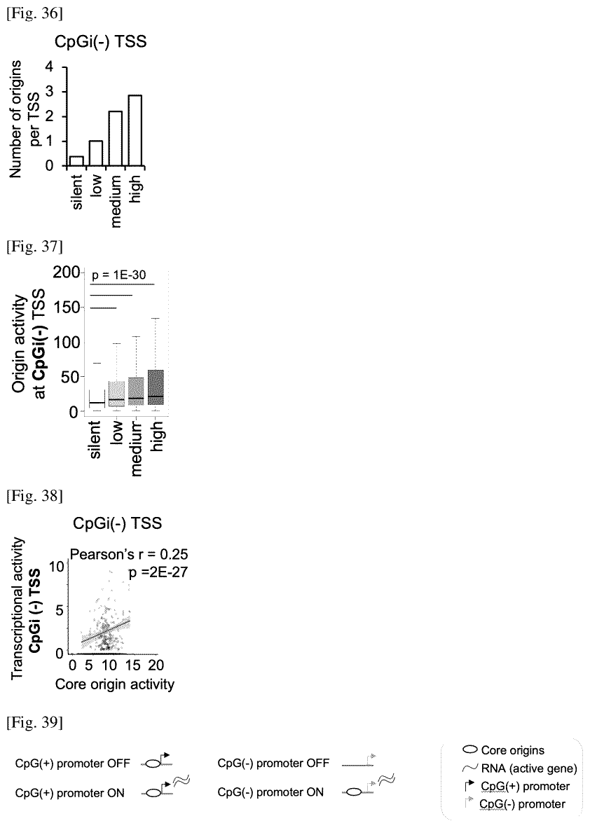

In contrast, there is a clear increase in origin positioning at CpGi(−) promoters when the level of transcription is increased (FIG. 35). Moreover, the number of clustered origins increased proportionally with the transcriptional activity, and the total origin activity was higher with increasing transcriptional activity (Pearson's correlation r=0.25—FIGS. 36, 37, 38). The inventors observed similar trends for gene promoters that contained a G-rich stretch of DNA instead of a CpGi (FIG. 82).

Immortalization Results in Increased Origin Positioning Stochasticity

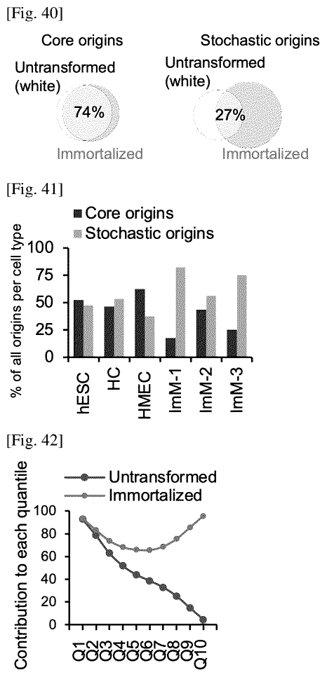

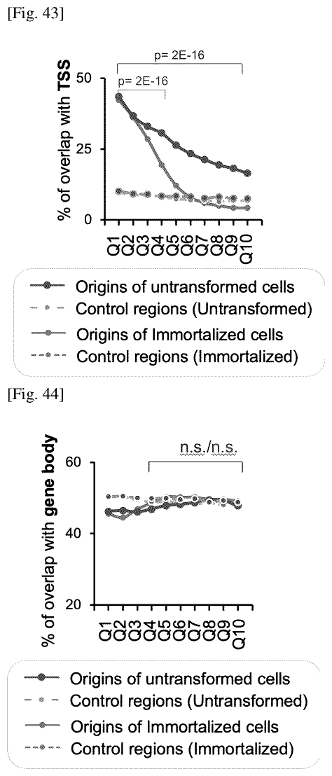

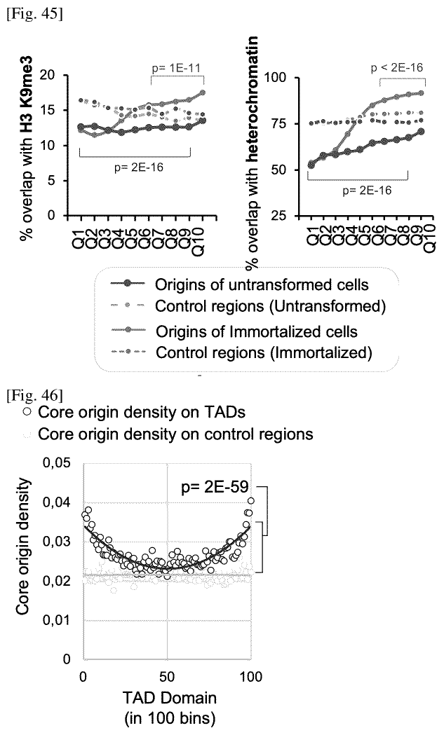

As aberrant DNA replication is a hallmark of many cancer cells, the inventors next asked whether the origin repertoire was disturbed after cell immortalization, a key step in cancer development leading to uncontrolled cell proliferation. To this aim, the inventors used three previously described immortalized cell lines obtained by mis-expression of oncogenes of the parental Human Mammary Epithelial Cell (HMEC) cell line: (i) ImM-1 in which p53 levels was reduced by at least 50% (ΔTP53), (ii) ImM-2 in which the oncogene RAS is overexpressed, and (iii) ImM-3 in which WNT is overexpressed. The inventors identified more origins in the immortalized cell types than in the untransformed cell types (hESC, HC and HMEC) (on average 100,000 vs 70,000 origins). This could not be due to higher proliferation rates in these cells as the hESC and HCs proliferated at the same or higher levels (see Methods). Nevertheless, untransformed and immortalized cell types shared a common core origin repertoire (FIG. 40) and the bulk of initiation events (˜80%) originated from core origins (FIG. 83). The higher number of origins in immortalized cells was clearly caused by an increase in stochastic origins (FIG. 41). While core (Q1 and Q2) origins were shared between untransformed and immortalized cell types, quantiles with lowest activity (Q8-10) were predominantly contributed by immortalized cell types (FIG. 42). In order to study origins from untransformed and immortalized cell types disjointedly, the inventors re-classified origins of each category into quantiles separately as described before. Genomic localization of core origins in relation to genes was comparable in untransformed and immortalized cell lines (FIGS. 43 and 44). However, stochastic origins from immortalized cells were less enriched near promoter regions (FIG. 44), but were enriched in heterochromatic regions (marked by K9me3) (FIG. 45). Therefore, immortalization induces low activity origins associated with what is heterochromatin in untransformed cells.

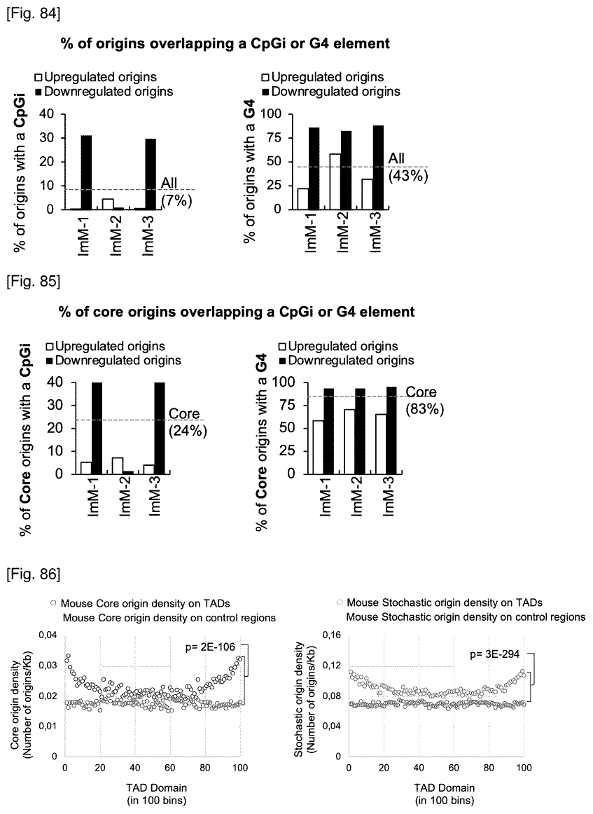

Immortalization also results in differentially up- or down-regulated origins. Strikingly, most down-regulated origins contain G-rich elements such as CpGi/G4, whereas up-regulated origins tend to be G-poor (FIGS. 84 and 85). Therefore, a change in the specification of origins occurs, with preference shifting from G-rich to G-poor DNA for both core and stochastic origins.

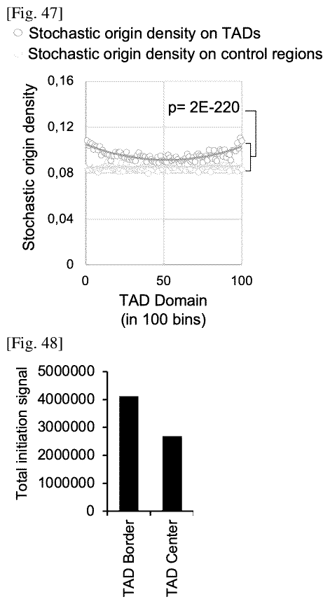

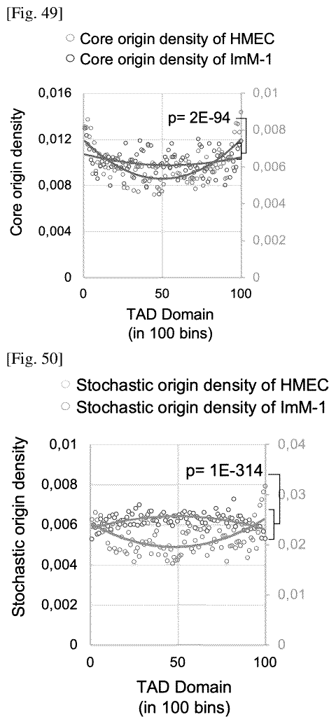

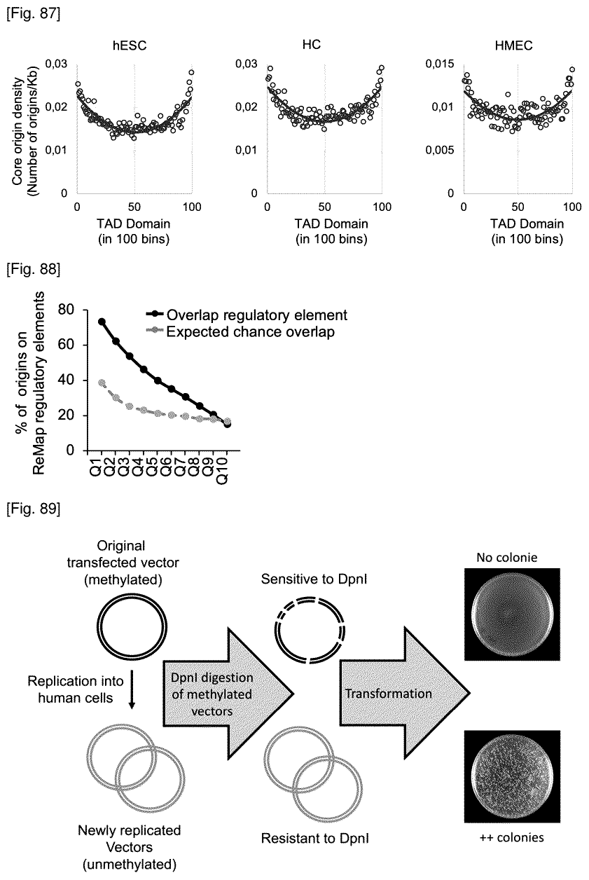

The inventors next asked whether there was a specific distribution of core and stochastic origins across topologically associating domains (TADs), which are large regions of the genome that self-interact to form three-dimensional (3D) structures. TAD borders are involved in the insulation of the corresponding chromatin domains, confining chromatin loops inside the TADs, and are enriched in TSS and the insulator factor CTCF. Both human core (FIG. 46) and stochastic origins (FIG. 47) were significantly enriched at TAD borders (i.e., “smiley” trend-line). Total amount of DNA replication initiation measured by SNS-seq was also 1.5 fold higher at TAD borders than at TAD center (FIG. 48). The inventors obtained similar results for mouse core and stochastic origins (FIG. 86). The inventors conclude that the replication origin density pattern mimics the structural organisation of the genome in individual chromatin domains. This distribution was clearly disturbed in immortalized ImM-1 (TP53^{KD}) cells compared with the parental HMEC cell line, and that this variation in origin density on TAD borders was statistically significant (FIGS. 49 and 50). Total amount of replication initiation at TAD borders and TAD centre was also markedly different in the ImM-1 cells compared to the parental HMEC (FIG. 51). hES cells, or other untransformed cell types did not display altered core origin density at TAD borders, suggesting that this property is specific to immortalization and does not reflect high proliferation rates (FIG. 87).

Altogether, these data suggest that the presence of either a CpGi/G-rich stretch or transcription is sufficient to recruit origin activity. In highly active promoters, CpGi or G-rich elements are not correlated with replication origin activity. Conversely, at inactive promoters CpGi/G-rich motifs are clearly associated with replication origin activity (summarised in FIG. 39). This result is also in line with the presence of G-rich elements at most replication origins.

### DISCUSSION

DNA replication origin specification remains poorly understood despite the progress in next-generation sequencing technology that allowed IS mapping genome-wide. In this study, the inventors used the SNS-Seq method, which has the highest resolution to map replication origins, in which the signal was corrected with suitable experimental controls generated in parallel (see Methods). The inventors found a remarkable consistency in the specification of a subset of IS, termed core origins, in multiple cell types that is maintained even after immortalization. Core origins, which represent −30,000 regions in any given cell type, hosted the bulk of DNA replication initiation events (70-85%) in all the studied cell types. The inventors uncovered that most core origins could be predicted by a computational algorithm based only on sequence recognition, thus unequivocally concluding that replication origins are preferentially activated in a precise set of regions in mammalian genomes in different cell types.

The inventors' study also reveals that the underlying DNA sequence is a prominent predictor of origin positioning in the human and mouse genomes. The G-rich sequence patterns commonly found in core origins were predictive of origin placement genome-wide. When present in the human genome, 72% of these patterns were associated with DNA replication initiation in at least one cell type. The stretch of G-rich repeated DNA sequence (OGRE) upstream of the IS corresponds with ORC1, ORC2 and MCM2-7 binding regions, coupled to a region with lower G and C content (FIGS. 19, 20, 21 and 22). Core origins are also often clustered, suggesting that they represent regions of the genome with several potential pre-RC binding sites. This organisation might constitute a broader pre-RC binding platform that may host several pre-RC and increase the efficiency of MCM loading and origin activation. Conversely, most stochastic origins contain a shorter stretch of G-rich region, possibly representing single putative pre-RC binding sites (FIG. 19). The position of the initiation sites revealed by SNS-seq is in perfect agreement with the positions of pre-RC factors determined independently, which are found upstream of the initiation site, coinciding with the G-rich region as expected, (FIG. 22). Importantly, this finding is an independent confirmation of the association of G-rich regions to metazoan replication origins.

How can a G-rich region be involved in initiation of DNA replication? One formal possibility for G-rich SNS-seq peaks could be the experimental protocol involving the use of lambda exonuclease, where G-rich sequences could be resistant to digestion (PMID: 25695952). However, the experimental conditions for SNS-seq used in most studies, including the inventors' ones but excluding the aforementioned study, are stringent (see Methods). Moreover, control SNS-seq samples treated in parallel (+RNase) are only slightly enriched in G-rich DNA. In addition, the G-rich nature of replication origins has been also confirmed using a nascent strand purification method that does not employ lambda exonuclease. Finally, some factors involved in initiation of DNA replication co-localize with DNA replication origins (this study) and can bind to G4 (see below).

A second possibility may be linked to the ON/OFF stages of DNA replication origins. The opening of DNA at the replication initiation sites requires two temporally successive steps. First, Pre-RCs form in G1, through the binding of ORC, Cdc6, Cdt1, which permit the recruitment of the MCM helicase. It is accepted that all potential origins are pre-set at this stage, but it is still not known how the metazoan origins are recognized by the ORC. The activation of the MCM helicase occurs at the G1-S transition, but only 20-30% of the pre-RCs are activated in S phase. A fundamental characteristic of G4 is its ability to form several structures, including folded and unfolded forms. These two forms might regulate the OFF stage (pre-RC) or the ON stage (initiation) of a replication origin; Exogenous G4 sequences able to form G4 structures do not inhibit the formation of pre-RCs in Xenopus egg extracts, but do compete with the firing of replication origins. This result may suggest that the folded form of G4 participates in the initiation of DNA synthesis but is not required for origin recognition by pre-RC proteins. In agreement, MTBP, RecqL and Rift, three factors involved in origin firing, all bind to G4.

A third possibility is guided by the NS profile at replication origins which may suggest that G4 act as a transient pause of the replication fork initiating at replication origins. Several previous studies have reported the enrichment of G-rich regions 5′ to the initiation site and suggested a transient pause of the replication fork at the G4. This hypothesis suggests that the G-rich/G4 structures are folded when origins are activated and then unfolded through a mechanism imposing a transient pause of the progressing replication fork, a phenomenon similar to transcriptional pausing.

The finding that the underlying DNA sequence is predictive of origin placement in a given species naturally leads to question to which extent chromatin and transcriptional environment is also involved in initiation of DNA replication. Origin positioning has previously been correlated with open chromatin and various histone marks related to active chromatin. Core origins often coincide with transcription and regulatory elements of the genome (e.g., promoters and enhancers) (FIG. 28, FIG. 88) that are associated with activating histone marks and open chromatin. It is conceivable that the DNA sequence pattern the inventors identified is usually part of open or permissive chromatin. However, core origins are also present in non-genic regions (19.4%) or silent genes. In addition, the impact of transcription and the presence of a G-rich element can be uncoupled. The presence of a G-rich element/CpGi in the promoter region of silent genes, or in non-coding regions, is sufficient to host replication origin activity. Of note, polycomb group proteins associate with CpGi(+) promoters and can bind to G4 DNA. The inventors previously showed that the presence of these proteins is a strong indicator of origin positioning, supporting a mechanism by which silent CpGi(+) gene promoters or repressed chromatin may host origins. Interestingly a recent report also supports a role for G4 elements in the regulation of polycomb-mediated gene repression. In conclusion, even though the DNA sequence information is not as strictly defined as the consensus ARS element sequence present at S. Cerevisiae origins, its predictive value shows that sequence specificity is a conserved feature of replication origins in metazoan cells. The inventors also acknowledge that a combination of select epigenetic marks together with sequence information might improve the prediction of metazoan replication origins.

Besides core origins, which represent most of the SNS signal, the inventors' analysis also identified thousands of stochastic origins, which poorly coincide with G-rich elements. Interestingly, immortalization greatly increased the number of these low-activity origins, especially within heterochromatic regions. This was accompanied by equalisation of DNA replication initiation events at TAD borders and centres (FIG. 51). The finding that replication origins are enriched at TAD borders might reflect a role for DNA replication origins in the formation of chromatin loops or their consequence. As such, density of origins could play a role in the insulation of replication domains. This is also reminiscent of previous findings that origin density/origin activity is highly correlated with replication timing. In addition, replication timing boundaries correlate with TAD boundaries. Hence, altered DNA initiation density, aberrant replication timing and altered chromosomal structure organisation might be linked in cell types undergoing immortalization. A previous study linked mis-expression of the oncogenes MYC and CCNE1 to formation of intragenic origins upon premature S-phase entry in a tumor-derived cell line. Here, the inventors show that both the number and distribution of replication origins is perturbed during immortalization, an important step in cellular transformation. Both the increased stochasticity in origin placement and perturbation of the DNA replication initiation density profile on TADs could therefore be new landmarks associated to cancer cells.

TABLE 1a

| | Percentage | | | | % of | | | |
|---|---|---|---|---|---|---|---|---|
| | of hg38 | | | | initiation | | | |
| | (number | | | | events | Of the | | |
| | of bases | | | | originating | origins | Number | % of |
| | that are | | | | from Core | shared | of origins | origins |
| | called | | | | origins | between | shared | shared |
| | origin/total | | | | (% of total | two cell | with at | with at |
| | number | Number of | Number of | % | SNS-seq | types, % | least | least |
| Number of | of bases | Core | Stochastic | Core | signal on | of Core | 1 other | 1 other |
| origins | in hg38) | origins | origins | origins | origins) | origins | cell type | cell type |
| 74534 | 1.3 | 39056 | 35478 | 52.4 | 72.9 | 81.1 | 57267 | 76.8 |
| 98086 | 1.5 | 45562 | 52524 | 46.5 | 79.9 | 82.1 | 61801 | 63.0 |
| 37703 | 0.7 | 23520 | 14183 | 62.4 | 87.2 | 84.3 | 31593 | 83.8 |
| 90761 | 1.0 | 15868 | 74893 | 17.5 | 73.2 | 65.7 | 39129 | 43.1 |
| 109137 | 1.9 | 47545 | 61592 | 43.6 | 85.0 | 79.2 | 63232 | 57.9 |
| 111531 | 1.4 | 27902 | 83629 | 25.0 | 78.6 | 70.2 | 55778 | 50.0 |
| 86958 | 1.3 | 33242 | 53716 | 41.2 | 82.2 | 77.1 | 51466.7 | 62.4 |

Number of DNA replication origins called per cell type (MACS2inSICER peaks, merged peaks from 2-6 replicates)

TABLE 1b

| | Nearest | | Origin name | Origin |
|---|---|---|---|---|
| Origin name | gene(s) | Reference | (this study) | type |
| LAMINB2 | LMNB2 | Giacca et al, PNAS, | HO_268397, | Core |
| | | 1994 | HO_268394 | |
| cMYC | MYC | Vassilev et al, MCB, | HO_146581 | Core |
| | | 1990 | | |
| MCM4 | PRKDC/ | Ladenburger et al, | HO_139765 | Q4 |
| | MCM4 | MCB, 2002 | | |
| HSP70 | HSPA1A | Taira et al, MCB 1994 | HO-104401 | Core |
| SCA-7 | ATXN7 | Nenguke, HMG, 2003 | HO-56313 | Core |
| HD | HTT | Nenguke, HMG, 2003 | HO_69221 | Core |
| (Huntington's | | | | |
| disease) | | | | |
| TOP1 | TOP1 | Keller et al, JBC, | HO_289103 | Q4 |
| | | 2002 | | |
| DNMT1 | DNMT1 | Araujo, JBC, 1999 | HO-271898- | Core at |
| | | | HO271901 | promoter, |
| | | | | (Q6, Q3, |
| | | | | Q4, Q1) |

Genomic coordinates of previously identified DNA replication origins (hg38)

TABLE 2a

| PREDICTOR | Description (based on 2 consecutive windows of 500 bp) |
|---|---|
| UP_A_fraction | Density of the base A in the first window (watson strand, 5′ to 3,) |
| UP_C_fraction | Density of the base C in the first window (watson strand, 5′ to 3,) |
| UP_G_fraction | Density of the base G in the first window (watson strand, 5′ to 3,) |
| UP_T_fraction | Density of the base T in the first window (watson strand, 5′ to 3,) |
| Down_A_fraction | Density of the base A in the second window (watson strand, 5′ to 3,) |
| Down_C_fraction | Density of the base C in the second window (watson strand, 5′ to 3,) |
| Down_G_fraction | Density of the base G in the second window (watson strand, 5′ to 3,) |
| Down_T_fraction | Density of the base T in the second window (watson strand, 5′ to 3,) |
| G_content_2 kb | Density of the base G 2 kb upstream from the first window |
| | (including) |
| C_content_2 kb | Density of the base C 2 kb downstream from the second window |
| | (including) |
| rampG | The slope with which the G-density drops from first to the second |
| | window) |
| rampC | The slope with which the C-density drops from first to the second |
| | window) |
| CC | The density of the indicated k-mer in the first window (watson |
| | strand) |
| CCC | The density of the indicated k-mer in the first window (watson |
| | strand) |
| CG | The density of the indicated k-mer in the first window (watson |
| | strand) |
| CGCG | The density of the indicated k-mer in the first window (watson |
| | strand) |
| GG | The density of the indicated k-mer in the first window (watson |
| | strand) |
| GGG | The density of the indicated k-mer in the first window (watson |
| | strand) |
| AA | The density of the indicated k-mer in both windows (watson strand) |
| AAA | The density of the indicated k-mer in both windows (watson strand) |
| TT | The density of the indicated k-mer in both windows (watson strand) |
| TTT | The density of the indicated k-mer in both windows (watson strand) |

Predictors used for machine learning in this study

TABLE 2b

| LR | | SVM | |
|---|---|---|---|
| PREDICTOR | weight | PREDICTOR | weight |
| UP_A | 0.0254 | UP_A | 0.218680435 |
| UP_C | 7.9 | UP_C | 0.139793978 |
| UP_G | 100 | UP_G | 9.371271338 |
| UP_T | 0.0249 | UP_T | 0.341651336 |
| DOWN_A | 0.0587 | DOWN_A | 0.873924681 |
| DOWN_C | 0.0306 | DOWN_C | 0.008394576 |
| DOWN_G | 0.044 | DOWN_G | 3.551440913 |
| DOWN_T | 0.087 | DOWN_T | 0.02648294 |
| G_2 kb | 0.594 | G_2 kb | 10.16243823 |
| C_2 kb | 0.012 | C_2 kb | 0.070957798 |
| rampG | 0.4332 | rampG | 6.94E−05 |
| rampC | 0.0026 | rampC | 4.29E−06 |
| AA | 0.1215 | AA | 5.25E−06 |
| AAA | 0.342 | AAA | 0.005761185 |
| CC | 0.0062 | CC | 0.000142966 |
| CCC | 0.6531 | CCC | 0.015779588 |
| CG | 0.1746 | CG | 0.002986597 |
| CGCG | 0.062 | CGCG | 0.107479555 |
| GG | 0.0528 | GG | 2.49E−05 |
| GGG | 0.0133 | GGG | 0.003187274 |
| TT | 0.0548 | TT | 8.57E−06 |
| TTT | 0.3173 | TTT | 0.008014669 |

Predictors used for machine learning in this study

TABLE 3

| Data | Cell Line |
|---|---|
| ORC1 ChIP-seq peaks | HeLa |
| ORC2 ChIP-seq peaks | K562 |
| MCM7 ChIP-seq peaks | HeLa |
| Gencode genes | not applicable |
| SNS-seq peaks (other study) | HeLa, K562, IMR90 (merged) |
| Phastcon20way scores | not applicable |
| H3K9me3 ChIP-seq peaks | H1 hESC |
| Heterochromatin | H1, K562 |
| INI-seq | in vitro, HeLA |
| OK-seq | HeLA |
| G4 mismatch | in vitro |
| G4H | human genome |
| TAD domains | human (hESC H1), mouse ESC |
| mappability | hg38 |
| Early and late replicating regions | H9 (hESC), Hematopoietic cells CD34+ |

Sources of datasets used in this study

TABLE 4

| | | Function/ | | |
|---|---|---|---|---|
| | | target | | |
| | Neighbouring | of | | |
| | gene | the | | |
| Primer | (if | primer | Forward | Reverse |
| pair | present) | pair | primer | primer |
| 1 | LMNB2 | origin | CACATGGAGGTTCTATG | CAAGTTCACGCCCAAGTA |
| | | | ACTGC (SEQ ID NO: | CA (SEQ ID NO: 43179) |
| | | | 43178) | |
| 2 | HBA1 | origin | GTCCACCCCTTCCTTCC | TGGAGGAGGTGAGACTT |
| | | | TC | AAGGA |
| | | | (SEQ ID NO: 43180) | (SEQ ID NO: 43181) |
| 3 | NPRL3 | origin | GAGTTCCGCGGTGCTGT | AACCAACATCGAGAGGG |
| | | | C (SEQ ID NO: 43182) | ACG (SEQ ID NO: 43183) |
| 4 | PAPD4 | origin | TGGGAGGTTCCAGCAGT | CCTCTTTTGGTCCTGGAG |
| | | | ATC (SEQ ID NO: 43184) | TG (SEQ ID NO: 43185) |
| 5 | DACH1 | origin | GAACTCGGAGCAGAGAC | GATGATCTCCCTCTCCTT |
| | | | TCC (SEQ ID NO: 43186) | TTCC (SEQ ID NO: 43187) |
| 6 | BTBD2 | origin | ACGGAGGGGTCACCAGT | CCCAACCCACTGTTTCTA |
| | | | AG (SEQ ID NO: 43188) | GG (SEQ ID NO: 43189) |
| 7 | LMNB2 | Background | GATTGAAAAGTCTCCGG | CGAACTGCCAGAACGTG |
| | | (no | GGC (SEQ ID NO: 43190) | TG (SEQ ID NO: 43191) |
| | | origin) | | |
| 8 | HBA1 | Background | GGGCTGACTTTCTCCCT | ACTCCACTCCCGCCCATC |
| | | (no | CG (SEQ ID NO: 43192) | (SEQ ID NO: 43193) |
| | | origin) | | |
| 9 | NPRL3 | Background | GAAGGCAGATCACGAGG | TCAAGCGATTCTCCTGTC |
| | | (no | TCA (SEQ ID NO: 43194) | CC (SEQ ID NO: 43195) |
| | | origin) | | |
| 10 | PAPD4 | Background | GGCAGGATTTAGGAACT | TCAGGATTCTTTAGAAAG |
| | | (no | GGA (SEQ ID NO: 43196) | CAGAAT (SEQ ID NO: |
| | | origin) | | 43197) |
| 11 | DACH1 | Background | AGGGAAATGAAACAGGG | GGGTCAGAAATAAATCCC |
| | | (no | ACA (SEQ ID NO: 43198) | CATAG (SEQ ID NO: |
| | | origin) | | 43199) |
| 12 | BTBD2 | Background | CCAGTGTGGGTGACAGA | GGACAGTGTGACCGAGG |
| | | (no | GTG (SEQ ID NO: 43200) | AGT (SEQ ID NO: 43201) |
| | | origin) | | |
| 13 | cMYC | origin | ACCAAGACCCCTTTAACT | CCTCGTCGCAGTAGAAAT |
| | | | CAAGA (SEQ ID NO: | ACG (SEQ ID NO: 43219) |
| | | | 43218) | |
| 14 | none | origin | TCTCACAGCTTGTGCAG | GCTGTTTCCCCACAAAAC |
| | (intergenic | | TCC (SEQ ID NO: 43202) | AC (SEQ ID NO: 43203) |
| | origin) | | | |
| 15 | none | origin | AGCCACGTTAGGGAAAG | CAAATGTGTTTCTTGGGT |
| | (intergenic | | GTC (SEQ ID NO: 43204) | TGG (SEQ ID NO: 43205) |
| | origin) | | | |
| 16 | none | origin | GCTGGAGTGGAGACAGT | CTCAAACCCAAACCCAAT |
| | (intergenic | | GAA (SEQ ID NO: 43206) | C (SEQ ID NO: 43207) |
| | origin) | | | |
| 17 | none | origin | TCTTGCTTTCTCCTTGCT | CAGGGGAGGTGAACAGA |
| | (intergenic | | GA (SEQ ID NO: 43208) | TG (SEQ ID NO: 43209) |
| | origin) | | | |
| 18 | none | Background | CAAGAATCGGACGTGAA | ATCATTCCAGGAATCCTC |
| | | (no | GG (SEQ ID NO: 43210) | TGG (SEQ ID NO: 43211) |
| | | origin) | | |
| 19 | none | Background | AGGGCTGAGCCATAATT | CTGCAATGCACTCACAAC |
| | | (no | CTTCT (SEQ ID NO: | AAC (SEQ ID NO: 43213) |
| | | origin) | 43212) | |
| 20 | none | Background | CTTGCACAATGCCTCAC | GAAAACACCAGCCACCA |
| | | (no | TCA (SEQ ID NO: 43214) | GAA (SEQ ID NO: 43215) |
| | | origin) | | |
| 21 | none | Background | GCTACTGATTCGGTGAG | GAGTTAAAGCACCCCTGT |
| | | (no | CAG (SEQ ID NO: 43216) | TGG (SEQ ID NO: 43217) |
| | | origin) | | |

List of primers used in this study (5 to 3 prime)

TABLE 5

| | | GS | GS + LR | GS + SVM |
|---|---|---|---|---|
| | | (overlapping | (overlapping | (overlapping |
| | Description | windows) | windows) | windows) |
| TPR | True positive rate | 0.51 | 0.36 | 0.34 |
| TNR | True negative rate | 0.87 | 0.97 | 0.98 |
| PPV | Positive predictive value TP/(TP + FP) | 0.20 | 0.36 | 0.34 |
| NPV | Negative predictive value | 0.97 | 0.96 | 0.96 |
| | TN/(TN + FN) | | | |
| BA | Balanced accuracy | 0.69 | 0.67 | 0.66 |
| | (0.5*(TP/(TP + FN) + TN/(TN + FP) | | | |

Confusion table displaying the performance of the genome scan (GS) and the machine learning algorithms on the test set.

### Example 2—Non-Viral Eukaryotic Vectors with Autonomous Replication

I. Main Objective

The goal of the inventors was to develop non-viral, self-replicating eukaryotic therapeutic vectors by introducing sequences containing a human origin of replication with high replicative capacity into defined plasmids. The sequences containing origins of replication of interest are previously determined through the exhaustive analysis of the repertoire of origins of replication of the human genome established in the laboratory.

II. Results

Objective 1: Define the minimum size and characteristics of vectors.

The first objective of this project was to define the basic receptor vector for insertion of our replication origins, as well as a rapid vector replication detection test.

1. DpnI Replication Test

This assay is based on the resistance of plasmids to digestion by DpnI, a methylated DNA digesting enzyme. (FIG. 89). The plasmids are prepared in E. Coli Dam+ bacteria. Therefore, the original plasmids used are methylated and sensitive to digestion by the restriction enzyme DpnI. In contrast, the DNA loses its methylation upon replication in human cells, and thus loses its sensitivity to DpnI. The replication status of the transfected plasmids can then be identified by testing its sensitivity to DpnI digestion. After transfection into bacteria, the formation of colonies indicates the presence of replicated plasmids (FIG. 89).

2. Basic Vector: pEPi-Del (peGFP-S/MAR)

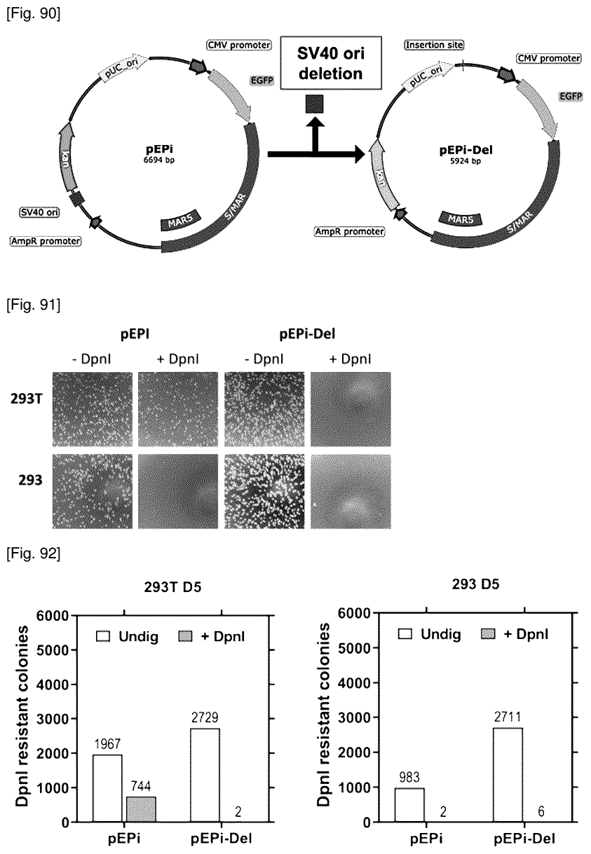

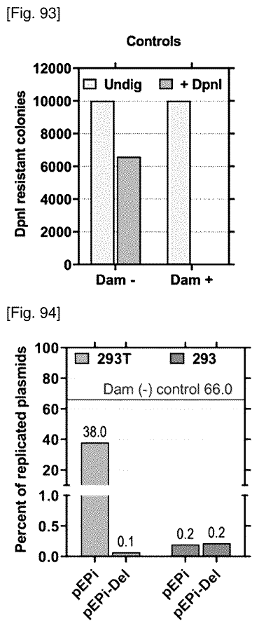

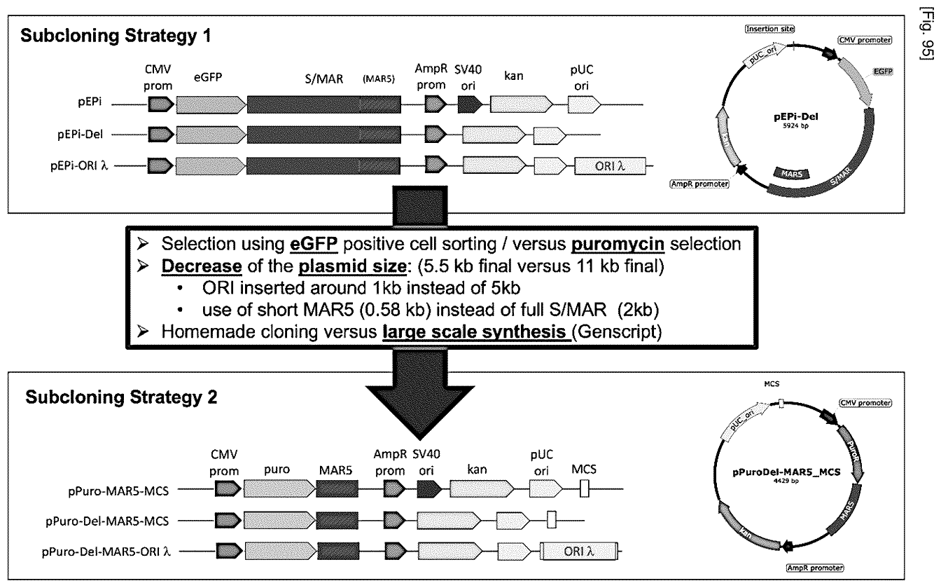

As a first step, the inventors tested the pEPi vector, a non-integrating vector whose expression can be monitored by fluorescence and which has the advantage of having an attachment site on the nuclear matrix allowing it to be better retained in the cell nucleus. The inventors had previously adapted it by removing the origin of replication of the SV40 virus that it contained (Ori SV40): pEPI-Del (FIG. 90). These two vectors allowed the inventors to develop their method for rapid testing of episomal replication in a dual cell system, HEK293T cells that express the large T antigen and allow replication of the SV40 origin (as a control) and HEK293 cells that do not express this antigen and do not allow replication of the SV40 virus origin (FIGS. 90-94).

Following the inventors' preliminary results, they readapted their strategy (FIG. 95). First, the inventors modified the reporter gene (eGFP) with a gene allowing antibiotic selection (puromycin) of positively transfected human cells. They also decreased the size of the S/MAR site. On the other hand, the inventors chose to be able to quickly screen a large number of sequences. The original sequences to be inserted were synthesized and cloned into the new receptor vector, using the assistance of the company Genscript.

3. Base Vector: pPuro-Del-MAR5

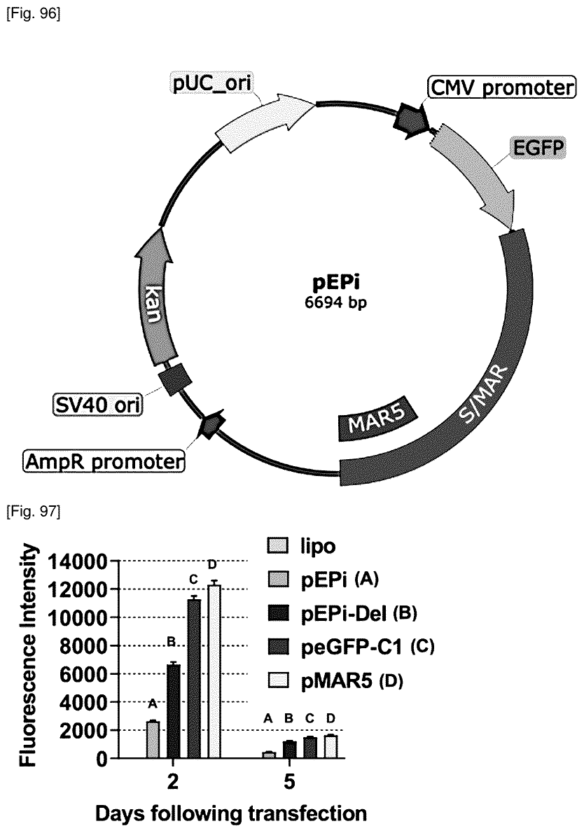

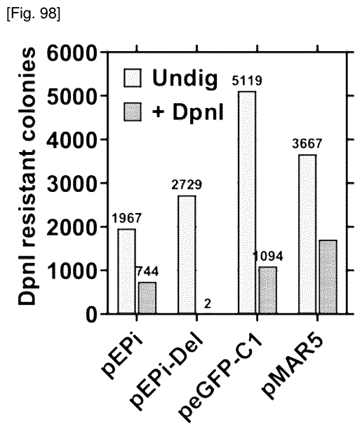

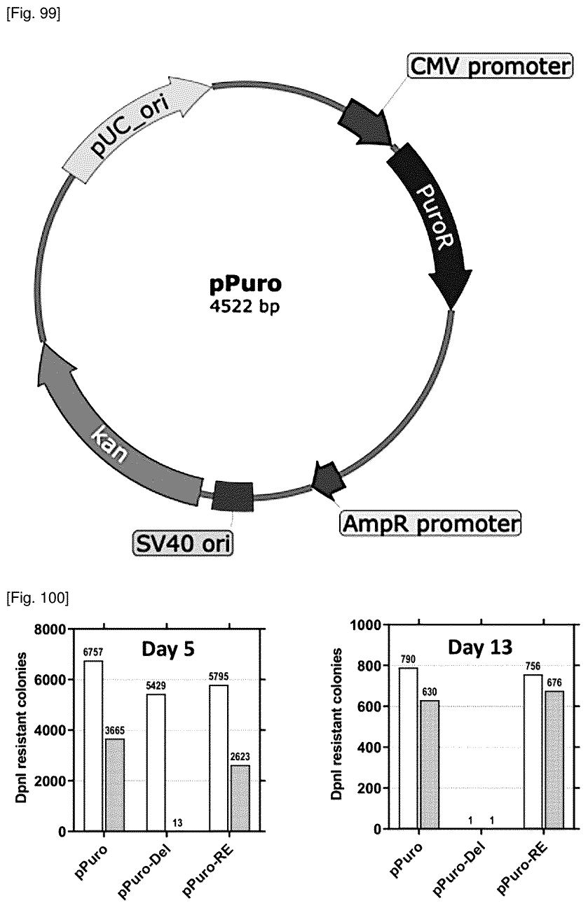

In order to validate the relevance of the inventor's new vector design, they first checked the impact of replacing the S/MAR sequence by the shorter MAR5 sequence (FIG. 96), as well as the impact of using the puromycin resistance gene instead of the one allowing eGFP expression (FIG. 99). The expression of eGFP was monitored by flow cytometry (FIG. 97). It shows that the vector with the MAR5 sequence (pMAR5) transfects 5 to 6 times better than the vector with the full S/MAR sequence, and as well as a vector with no nuclear matrix binding sequence (peGFP-C1). The replication assay (FIG. 98) shows a higher replication rate of the pMAR5 plasmid than the vector with the S/MAR (pEPi) and higher than the pEGFP-C1 vector. These results demonstrate the value of a reduced S/MAR sequence size. Furthermore, the replacement of the eGFP sequence with the gene conferring puromycin resistance allows the use of the Dpn1 replication assay up to at least 13 days after cell transfection, compared to 5 days with the previous construct (FIG. 100). The receptor vector finally retained and cloned: pPuroDel-MAR5_MCS is presented in FIG. 102.

Objective 2: Qualitative and quantitative analysis of autonomous replicative capacity (WP 2.1).

1. Selection and Synthesis of the Origin Bank to be Tested

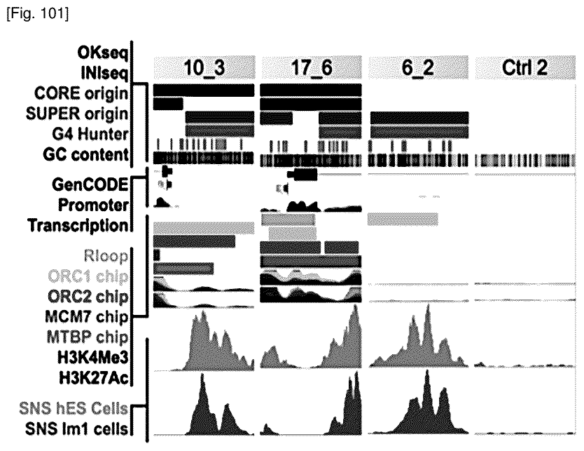

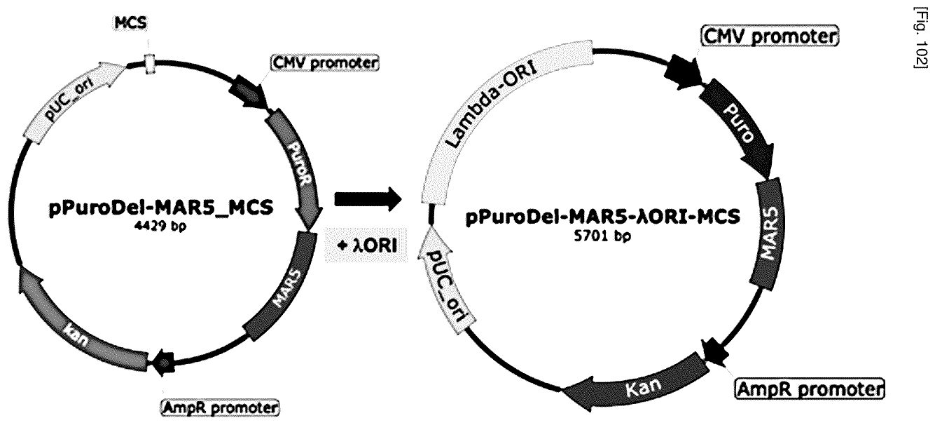

The inventors selected 67 sequences containing human replication origins and 2 control sequences (synthesized by the company Genscript). These sequences were chosen in view of the method according to the invention, i.e. the complete repertoire of replication origins identified by the inventors. A genome-wide and high-resolution repertoire of human genome replication origins was identified by an analysis of 24 triplicate samples obtained from different human cell types: pluripotent embryonic stem cells, primary CD34 cells, hematopoietic differentiating CD34 cells, epithelial cells, and oncogene immortalized epithelial cells. This analysis revealed a particular class of origins that we named “Core origins” (Core Oris) which are responsible for 80% of the replication initiation signal, and which are common to most of the cell types analyzed. the inventors have selected a series of origins that present different characteristics representative of CORE origins. These criteria are for example the presence of binding sites of the ORC complex proteins involved in the recognition of origins, the frequency of sites capable of forming G quadruplexes (G4), the presence of transcription initiation sites (TSS), the presence of post-translational modifications of Histone 3 (e.g. H3K4Me3), the presence of Rloop, the co-validation of the location of these origins by other techniques (IniSeq, EdUseq), the presence of binding sites of the Treslin-MTBP complex which is involved in the activation of the helicase responsible for the initiation of replication 4 examples of origin profiles are presented (FIG. 101).

Sequences were cloned into pPuro-Del-MAR5-MCS at the EcorV site contained in the multiple cloning site (MCS) (FIG. 102). Upon receipt of the library (i.e. containing the origins), the vectors were transformed into competent bacteria, subcloned, and then prepared. Their overall size and structure were verified by restriction enzyme digestion followed by agarose gel migration. In addition to the expected profiles of “simple” vectors, we identified dimeric plasmids (or mix of simple and dimeric plasmids) that we had to simplify in order to continue our study (about ¼ of the library).

2. Application of the Dpn1 Assay to the Vector Library

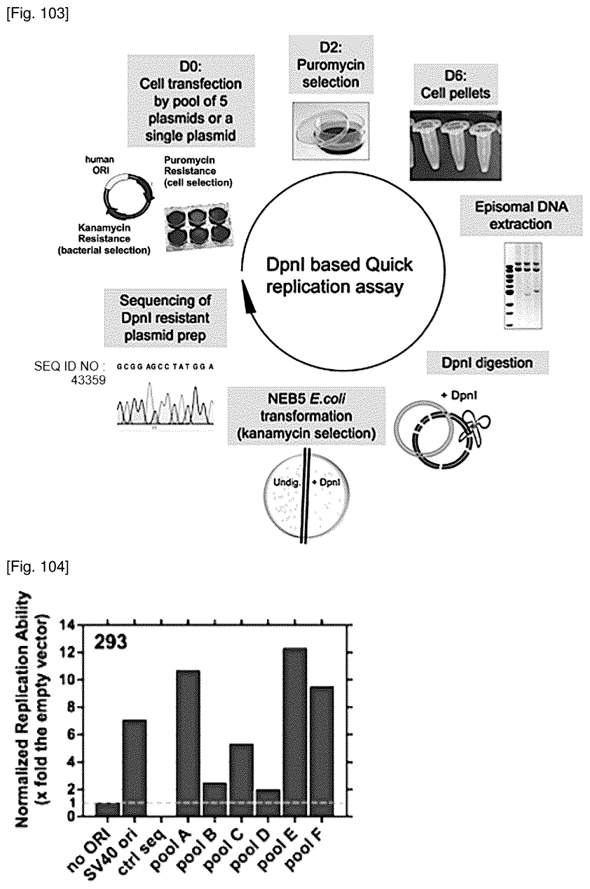

To assess the autonomous replication capacity of the vectors from the library, we applied our rapid replication assay based on DpnI digestion to 293T or 293 cells transfected with pools of 5 plasmid vectors (FIGS. 103 and Table 6). At the end of the assay, the colonies were counted and the results of the replication capacity of the plasmids (6 days after transfection) were presented (FIG. 104). The plasmids contained in the kanamycin-resistant colonies from the DpnI digestion were prepared and sequenced. Once identified, vectors that were able to replicate autonomously were individually resubmitted to the rapid replication assay. 6 days after transfection, replication is clearly detected. However, its rate is low compared to a vector containing the SV40 origin of replication, in 293T cells encoding the viral replication protein (T antigen). However, SV40 has the ability to deregulate the cell cycle and allows viral DNA to be re-replicated within the same cell cycle. This is totally impossible for cell replication origins, a major regulation of which is that each origin can only be used once and only once during the same cell cycle. Indeed, re-replication leads to gene amplification phenomena resulting in genomic instability. The inventors have undertaken a quantification by qPCR or ddPCR as well as an evaluation at later times (12-13 days after transfection) in order to estimate more precisely the number of vectors replicated during successive cell divisions. These data demonstrate that the replication origins allow a self-replication of the vectors comprising them in Eukaryotic cells.

TABLE 6

| Pool | Vectors |
|---|---|
| A | 1_5 |
| | 2_1 |
| | 2_2 |
| | 3_1 |
| | 3_2 |
| B | 3_3 |
| | 3_4 |
| | 6_4 |
| | 6_5 |
| | 6_6 |
| C | 6_7 |
| | 8_1 |
| | 8_2 |
| | 8_3 |
| | 8_4 (c-Myc) |
| D | 11_3 |
| | 11_4 |
| | 14_3 |
| | 16_3 |
| | 17_4 |
| E | 17_5 |
| | 17_6 |
| | 19_2 |
| | 19_3 |
| | 19_4 |
| F | 19_5 |
| | 19_6 |
| | 19_7 |
| | 19_8 |
| | 19_9 |
| SV40 ori | pPuro5-RE-SV40 |
| No ORI | pPuro5-Del-MCS |
| Ctrl Seq | Ctrl2 |

3. Special Cases of Replication of Dimeric Vectors

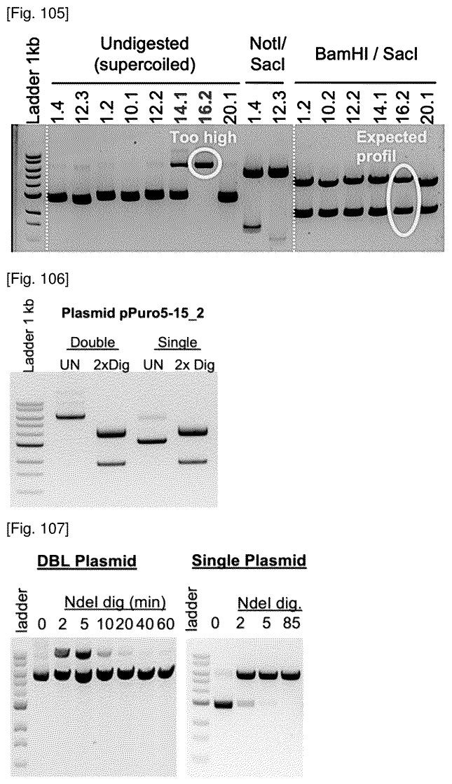

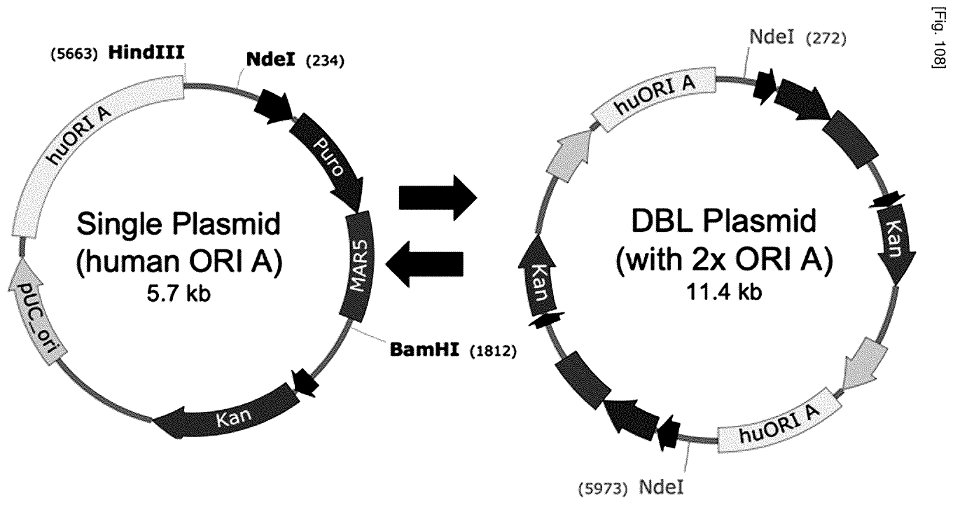

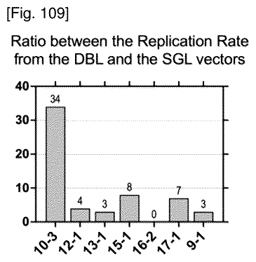

During the subcloning of the vector library, the inventors highlighted the presence of dimeric vectors, symmetrical (FIG. 108), showing a band profile of the supercoiled form of the plasmid, 2 times higher than expected, while the double digestion profile is the one expected for a single plasmid (FIG. 105, for instance 16.2). In other cases, the inventors observed plasmid preparations containing both the single and double forms (case of 14.1, FIG. 105). Partial digestion of these vectors with a restriction enzyme cutting a single site of the single vector (example, 15.2, FIGS. 106 and 107) confirms the dual size of the dimeric plasmids. Interestingly, the inventors observed that dimeric plasmids have a better replication capacity than their simple form (FIG. 109) (especially for vector 10.3). This observation motivates the production of vectors containing multiple origins, when necessary.

4. Sequence of the Vectors
empty vector (without human Origin) pPuroDel-MAR5_MCS: SEQ ID NO: SEQ ID No: 43289.

The following vectors contain an origin of replication as defined in the present invention:
>1_1_pPuroDel-MAR5_MCS: SEQ ID NO: 43290
>1_2_pPuroDel-MAR5_MCS: SEQ ID NO: 43291
>1_3_pPuroDel-MAR5_MCS: SEQ ID NO: 43292
>1_4_pPuroDel-MAR5_MCS: SEQ ID NO: 43293
>10_1_pPuroDel-MAR5_MCS: SEQ ID NO: 43294
>10_2_pPuroDel-MAR5_MCS: SEQ ID NO: 43295
>10_3_pPuroDel-MAR5_MCS: SEQ ID NO: 43296
>10_4_pPuroDel-MAR5_MCS: SEQ ID NO: 43297
>11_1_pPuroDel-MAR5_MCS: SEQ ID NO: 43298
>11_2_pPuroDel-MAR5_MCS: SEQ ID NO: 43299
>12_1_pPuroDel-MAR5_MCS: SEQ ID NO: 43300
>12_2_pPuroDel-MAR5_MCS: SEQ ID NO: 43301
>12_3_pPuroDel-MAR5_MCS: SEQ ID NO: 43302
>13_1_pPuroDel-MAR5_MCS: SEQ ID NO: 43303
>14_1_pPuroDel-MAR5_MCS: SEQ ID NO: 43304
>14_2_pPuroDel-MAR5_MCS: SEQ ID NO: 43305
>15_1_pPuroDel-MAR5_MCS: SEQ ID NO: 43306
>15_2_pPuroDel-MAR5_MCS: SEQ ID NO: 43307
>15_3_pPuroDel-MAR5_MCS: SEQ ID NO: 43308
>15_4_pPuroDel-MAR5_MCS: SEQ ID NO: 43309
>16_1_pPuroDel-MAR5_MCS: SEQ ID NO: 43310
>16_2_pPuroDel-MAR5_MCS: SEQ ID NO: 43311
>17_1_pPuroDel-MAR5_MCS: SEQ ID NO: 43312
>17_2_pPuroDel-MAR5_MCS: SEQ ID NO: 43313
>17_3_pPuroDel-MAR5_MCS: SEQ ID NO: 43314
>18_1_pPuroDel-MAR5_MCS: SEQ ID NO: 43315
>19_1_pPuroDel-MAR5_MCS: SEQ ID NO: 43316
>20_1_pPuroDel-MAR5_MCS: SEQ ID NO: 43317
>21_1_pPuroDel-MAR5_MCS: SEQ ID NO: 43318
>5_1_pPuroDel-MAR5_MCS: SEQ ID NO: 43319
>6_1_pPuroDel-MAR5_MCS: SEQ ID NO: 43320
>6_2_pPuroDel-MAR5_MCS: SEQ ID NO: 43321
>6_3_pPuroDel-MAR5_MCS: SEQ ID NO: 43322
>7_2_pPuroDel-MAR5_MCS: SEQ ID NO: 43323
>9_1_pPuroDel-MAR5_MCS: SEQ ID NO: 43324
>9_2_pPuroDel-MAR5_MCS: SEQ ID NO: 43325
>9_3_pPuroDel-MAR5_MCS: SEQ ID NO: 43326
>1_5_pPuroDel-MAR5_MCS: SEQ ID NO: 43327
>11_3_pPuroDel-MAR5_MCS: SEQ ID NO: 43328
>11_4_pPuroDel-MAR5_MCS: SEQ ID NO: 43329
>14_3_pPuroDel-MAR5_MCS: SEQ ID NO: 43330
>16_3_pPuroDel-MAR5_MCS: SEQ ID NO: 43331
>17_4_pPuroDel-MAR5_MCS: SEQ ID NO: 43332
>17_5_pPuroDel-MAR5_MCS: SEQ ID NO: 43333
>17_6_pPuroDel-MAR5_MCS: SEQ ID NO: 43334
>19_2_pPuroDel-MAR5_MCS: SEQ ID NO: 43335
>19_3_pPuroDel-MAR5_MCS: SEQ ID NO: 43336
>19_4_pPuroDel-MAR5_MCS: SEQ ID NO: 43337
>19_5_pPuroDel-MAR5_MCS: SEQ ID NO: 43338
>19_6_pPuroDel-MAR5_MCS: SEQ ID NO: 43339
>19_7_pPuroDel-MAR5_MCS: SEQ ID NO: 43340
>19_8_pPuroDel-MAR5_MCS: SEQ ID NO: 43341
>19_9_pPuroDel-MAR5_MCS: SEQ ID NO: 43342
>2_1_pPuroDel-MAR5_MCS: SEQ ID NO: 43343
>2_2_pPuroDel-MAR5_MCS: SEQ ID NO: 43344
>20_2_pPuroDel-MAR5_MCS: SEQ ID NO: 43345
>22_1_pPuroDel-MAR5_MCS: SEQ ID NO: 43346
>3_1_pPuroDel-MAR5_MCS: SEQ ID NO: 43347
>3_2_pPuroDel-MAR5_MCS: SEQ ID NO: 43348
>3_3_pPuroDel-MAR5_MCS: SEQ ID NO: 43349
>3_4_pPuroDel-MAR5_MCS: SEQ ID NO: 43350
>6_4_pPuroDel-MAR5_MCS: SEQ ID NO: 43351
>6_5_pPuroDel-MAR5_MCS: SEQ ID NO: 43352
>6_6_pPuroDel-MAR5_MCS: SEQ ID NO: 43353
>6_7_pPuroDel-MAR5_MCS: SEQ ID NO: 43354
>8_1_pPuroDel-MAR5_MCS: SEQ ID NO: 43355
>8_2_pPuroDel-MAR5_MCS: SEQ ID NO: 43356
>8_3_pPuroDel-MAR5_MCS: SEQ ID NO: 43357
>8_4_Myc_pPuroDel-MAR5_MCS: SEQ ID NO:

1-15. (canceled)

16. A method for isolating a mammalian genomic DNA replication origin, the method comprising:
(a) isolating the genomic DNA molecules from a somatic cell of a mammal;
(b) dividing the genomic DNA molecules into 500 bp windows every 100 pb along said genomic DNA molecules,
(c) identifying a first 500 bp windows such that:
the first 500 bp window has at least 172 G nucleotides,
the first 500 bp window has at least 105 A or T nucleotides,
a second 500 bp window immediately adjacent to the first 500 bp window at the 3′-end of the window has a G content lower than the 172 and higher than 125;
wherein the variation of the G content between the first and the second 500 bp window is ranging from 8% to 40%;
the G content in a large window consisting of 8 consecutive 500 bp-windows constituted by a third 500 bp windows adjacent to a fourth 500 bp windows, itself adjacent to a fifth 500 bp windows, itself adjacent to the first 500 bp windows, itself adjacent to the second 500 bp windows, itself adjacent to a sixth 500 bp windows, itself adjacent to a seventh 500 bp windows, itself adjacent to a eighth 500 bp windows, is higher than 960;
isolating from the genomic DNA molecules the fragments that have a size from 500 pb up 6000 pb corresponding to a putative mammalian genomic DNA replication origin, wherein the putative mammalian genomic DNA replication origin consists at its 5′ end of the first 500 bp window,
selecting from said putative mammalian genomic DNA replication origin a fragment that is able, when contained in the DNA of an Eukaryotic cell, to produce nascent DNA, and to initiate DNA replication; and
isolating said fragment, wherein said fragment is a mammalian genomic DNA replication origin.

17. The method for isolating a mammalian genomic DNA replication origin according to claim 16, wherein said putative mammalian genomic DNA replication origin have size varying from 500 bp to 4000 bp.

18. The method for isolating a mammalian genomic DNA replication origin according to claim 16, wherein the first 500 bp window of a fragment interacts with ORC1 or ORC2 replication initiation factors.

19. The method for isolating a mammalian genomic DNA replication origin according to claim 16, wherein sequence immediately adjacent to the first 500 pb window contains:
either multiple tandemly G4 structures, wherein said tandemly G4 structures are present up to 12 times, or
G-rich Repeated Element, or OGRE, or
both.

20. The method for isolating a mammalian genomic DNA replication origin according to claim 16, wherein the fragment contains a 716 pb core initiation origin sequence, the core initiation origin sequence being complementary to nascent DNA fragments sequence.

21. The method for isolating a mammalian genomic DNA replication origin according to anyone of claim 16, wherein the fragment contains polycomb proteins binding sites or histone acetylation marks, or both.

22. An isolated and purified mammalian genomic DNA replication origin liable to be obtained by the method as defined in claim 16, the mammalian genomic DNA replication origin comprising one of the sequences as set forth in SEQ ID NO: 1 and SEQ ID NO: 3 to SEQ ID NO: 43,177 and in SEQ ID NO: 43,220 to 43,288.

23. The isolated and purified mammalian genomic DNA replication origin liable to be obtained by the method as defined in claim 16, the mammalian genomic DNA replication origin consisting of one of the sequences as set forth in SEQ ID NO: 1 to SEQ ID NO: 43,177 and in SEQ ID NO: 43,220 to 43,288.

24. A vector comprising:
a mammalian genomic DNA replication origin as defined in claim 22,
at least a sequence coding for a protein allowing the resistance to a compound killing eukaryotic cells, and
a region independent to the mammalian genomic DNA replication origin allowing to insert a gene of interest and its expression.

25. The vector according to claim 24, further comprising
a prokaryotic replication origin.
a sequence coding for a protein allowing the resistant to an antibiotic.

26. The vector according to claim 24, comprising or consisting in a sequence acid sequence as set forth in SEQ ID NO: 43,290 to 43,358.

27. A mammalian cell comprising a vector as defined in claim 24.

28. A non-human mammal comprising a cell according to claim 27.

29. A method for expressing in a mammal cell a gene of interest, the method comprising administering a vector in the mammal cell, the vector being as defined in claim 24, the vecor comprising the gene of interest, the sequence of the gene of interest being inserted in the vector in the region independent to the mammalian genomic DNA replication origin.

30. A computer program product implemented on an appropriated support comprising instructions to execute the steps b- to c- of the method of claim 16.
